(19) United States
(12) Patent Application Publication
(10) Pub. No.: US 2008/0313770 A1
(43) Pub. Date: Dec. 18, 2008

(54) Methods and compositions for modulating tocol content

(75) Inventors: Knut Meyer, Wilmington, DE (US); Bo Shen, Johnston, IA (US); Mitchell C. Tarczynski, West Des Moines, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignees: Pioneer Hi-Bred International, Inc., Johnston, IA (US); E.I. du Pont de Nemours and Company, Wilmington, DE (US)

(21) Appl. No.: 11/655,654

(22) Filed: Jan. 19, 2007

Related U.S. Application Data
(60) Provisional application No. 60/761,168, filed on Jan. 23, 2006.

(51) Int. Cl. C12N 15/29; A01H 5/00

(52) U.S. Cl. 800/278; 800/298; 800/306; 800/322; 800/314; 800/320; 800/320.1; 800/320.2; 800/320.3

(57) ABSTRACT

Compositions comprising a modulated tocol content in a plant or plant part are provided. In specific embodiments, the compositions and methods of the invention modulate tocol content by modulating the level of a polypeptide having a LEC1-type B domain in combination with modulating the level of at least one other polypeptide involved in tocol biosynthesis. Plants, plant parts, grain, seed and oil having the modulated tocol level are also provided. Methods to enhance oxidative stress tolerance of a plant or plant part, increase shelf-life, enhance the nutritional value, and improve tissue quality are also provided.

### CROSS-REFERENCE TO RELATED APPLICATIONS

This application claims the benefit of U.S. Provisional Application No. 60/761,168, filed on Jan. 23, 2006, herein incorporated by reference in its entirety.

### FIELD OF THE INVENTION

The invention relates to the genetic modification of plants. In particular, methods and compositions are provided for modulating tocol content in a plant or plant part.

### BACKGROUND OF THE INVENTION

The vitamin E family of antioxidants in plants comprises tocotrienols and tocopherols. Each of these classes of compounds contains a polar chromanol ring linked to an isoprenoid-derived hydrocarbon chain. The structure of tocotrienols differs from that of tocopherols by the presence of three trans double bonds in the hydrocarbon tail. In addition, the α, β, γ, and δ species of both tocopherols and tocotrienols differ with regard to the numbers and positions of methods groups on the chromanol ring.

Tocotrienols and tocopherols can display a diversity of biological and physiological properties. For example, they are potent antioxidants and can protect plants against oxidative stresses. The potent lipid-soluble antioxidant properties of tocols further provide considerable nutritive value in human and animal diets and therefore contribute to the nutritive value of food products and animal feeds derived from cereal grains (Packer et al. (2001) J. Nutr. 131:369 S-373S, Andlaueer et al. (1998) Cereal Foods World 43:356-359 and Wang et al. (1993) Plant Foods 43:9-17). Tocols have also been linked to a number of beneficial therapeutic properties, including the ability to reduce serum cholesterol (Theriault et al. (1999) Clin. Biochem 32:309-319 and Raederstorff et al. (2002) Ann. Nutr. Metab. 46:17-23) and inhibit the growth of breast cancer cells (Nesaretnam et al. (1998) Lipids 33:461-469 and Elson et al. (1994) J. Nutr. 124: 607-614). Based on their health promoting properties, tocols are commercially produced as nutraceuticals.

Methods and compositions are needed in the art that allow the level of tocol content in a plant to be increased.

### BRIEF SUMMARY OF THE INVENTION

Methods and compositions are provided for modulating tocol content in a plant or plant part. In specific methods, tocol content in a plant or part thereof is increased by increasing the level of a polypeptide comprising a LEC1-type B domain or a biologically active variant or fragment thereof and modulating the level of a polypeptide involved in tocol biosynthesis. In further methods, the increase in tocol content comprises an increase in tocopherol content and/or tocotrienol content.

Compositions and methods are also provided which comprise grain or seed which comprise an increased level of tocol, tocopherol, and/or tocotrienol content. In specific embodiments, the grain or seed is from maize.

Methods of increasing tocol content, tocopherol content and/or tocotrienol in a plant or plant part, methods to improve the tissue quality of an animal, and various animal feeds are also provided.

### BRIEF DESCRIPTION OF THE DRAWINGS

FIG. 1 provides a sequence alignment of various members of the HAP3 transcriptional activator family. The alignment provides a consensus sequence (SEQ ID NO: 25) and also outlines domains A, B and C. The remaining aligned sequences include AtNF-YB-6 (LEC1-like) (SEQ ID NO:10); AtNF-YB-9(LEC1) (SEQ ID NO: 11); OsLEC1 (SEQ ID NO: 12); Zm_LEC1 (SEQ ID NO: 13); Zm_LEC1-like (SEQ ID NO: 14); ZmHAP3-2 (SEQ ID NO: 15); Soybean LEC1 (SEQ ID NO: 16); wheat LEC1 (SEQ ID NO: 17); AtNF-YB-4 (SEQ ID NO: 18); AtNF-YB-5 (SEQ ID NO: 19); Zm_HAP3L6 (SEQ ID NO: 20); Zm_HAP3L9 (SEQ ID NO: 21); Zm_HAP3L2 (SEQ ID NO: 22); OsHAP3L6 (SEQ ID NO: 23); and OsHAP3L9 (SEQ ID NO: 24).

FIG. 2 provides a sequence alignment of various LEC-1 type B domains (light shading) and non-LEC1 type B domains (dark shading). The aligned sequences include The remaining sequences aligned include AtNF-YB-6 (LEC1-like) (amino acids 57-92 of SEQ ID NO:10); AtNF-YB-9(LEC1) (amino acids 58-84 of SEQ ID NO: 11); OsLEC1 (amino acids 31-99 of SEQ ID NO:12); Zm_LEC1 (amino acids 36-100 of SEQ ID NO:13); Zm_LEC1-like (amino acids 32-98 of SEQ ID NO: 14); ZmHAP3-2 (amino acids 48-78 of SEQ ID NO: 15); Soybean LEC1 (amino acids 28-91 of SEQ ID NO: 16); wheat LEC1 (amino acids 23-78 of SEQ ID NO: 17); AtNF-YB-4 (amino acids 2-55 of SEQ ID NO: 18); AtNF-YB-5 (amino acids 50-64 of SEQ ID NO: 19); Zm_HAP3L6 (amino acids 28-64 of SEQ ID NO: 20); Zm_HAP3L9 (amino acids 22-63 of SEQ ID NO: 21); Zm_HAP3L2 (amino acids 35-73 of SEQ ID NO: 22); OsHAP3L6 (amino acids 33-64 of SEQ ID NO: 23); OsHAP3L9 (amino acids 34-60 of SEQ ID NO: 24) and the consensus sequence (amino acids 67-117 of SEQ ID NO:25).

FIG. 3 provides a sequence alignment of various members of the HGGT family. The aligned sequences include the consensus sequence (SEQ ID NO: 32); hv hggt.pro (SEQ ID NO:6); os hggt.pro (SEQ ID NO: 30) and t hggt.pro (SEQ ID NO:31).

FIG. 4 provides a sequence alignment of various members of the HPT family. The aligned sequences include the consensus sequence (SEQ ID NO:29); maize hpt.pro (SEQ ID NO:9); rice hpt BAD38343.pro (SEQ ID NO:26); soy hpt AAX56086.pro (SEQ ID NO:27); ath hpt AAL35412.seq.pro (SEQ ID NO:28).

FIG. 5 shows the effect of LEC1 on embryo γ-tocopherol content.

FIG. 6 shows the effect of LEC1 on embryo α-tocopherol content.

FIG. 7 shows the effect of LEC1 on whole grain tocopherol content. Tocol is tocopherol; T3 is tocotrienol; total is the sum of tocopherol and tocotrienol. Tocopherol content of three ears of transgenic LEC1 and null were presented.

FIG. 8 shows the effect of LEC1 on tocopherol pathway gene expression.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods for modulating tocol content in a plant or plant part are provided. The term “tocol” refers generally to any of the tocopherol (alpha, beta, gamma and delta) and tocotrienol (alpha, beta, gamma and delta) molecular species that are known to occur in biological systems. Such compounds comprise a series of related benzopyranols (or methyl tocols) including tocopherols and tocotrienols. Tocopherols have a saturated C16 side chain, and the tocotrienols have an unsaturated C16 side chain with three double bonds. The four main constituents of tocols are termed alpha, beta, gamma and delta. See, for example, IUPAC-IUB JCBN (1982) Arch. Biochem. Biophys. 218: 347-348; IUPAC-IUB JCBN (1982) Eur. J. Biochem. 123: 473-475; IUPAC-IUB JCBN (1982) Mol. Cell. Biochem. 49: 183-185; Liébecq (1982) Pure Appl. Chem. 54: 1507-1510; Biochemical Nomenclature and Related Documents (1992) 2nd edition, Portland Press: 239-241, each of which is herein incorporated by reference.

“Tocotrienols” as used herein, refer to any individual tocotrienol or any mixture of two or more tocotrienols. The mixture may contain other components, including tocopherols. “Tocopherols” as used herein, refer to any individual tocopherol or any mixture of two or more tocopherols. The mixture may contain other components, including tocotrienols.

The term “tocol level” refers to the total amount of tocopherol and tocotrienol in a whole plant, plant part, plant tissue (seed, kernel, or grain) or plant cell or in a microbial host. The term “tocol composition” refers both to the ratio of the various tocols produced in any given biological system and to altered characteristics, such as antioxidant activity, of any one tocol compound.

“Modulating tocol content” includes any decrease or increase in the total tocol level and/or the tocol composition in a whole plant, plant part, plant tissue, plant cell or microbial host. For example, modulating tocol content can comprise either an increase or a decrease in overall tocol level of about 0.1%, 0.5%, 1%, 5%, 10%, 20%, 30%, 40%, 50%, 60%, 70%, 80%, 90%, 100%, 120% or greater when compared to a control plant or plant part. Alternatively, the modulated tocol level can include about a 0.5 fold, 1 fold, 2 fold, 4 fold, 8 fold, 16 fold, 32 fold or greater overall increase or decrease in tocol level in the plant or the plant part when compared to a control plant or plant part.

Moreover, the modulation of the tocol content can also include a modulation in tocol composition: a change in the ratio of one or more tocols and/or the altered characteristic of one or more tocol. For example, the ratio of various tocols such as the alpha, beta, gamma and/or delta tocotrienols and/or tocopherols could be altered and thereby modulate the tocol content of the plant or plant part when compared to a control plant. In specific embodiments, the ratio of tocotrienol to totocopherol is altered.

Similarly, the tocotrienol content (i.e., tocotrienol composition and/or level) or the tocopherol content (i.e., tocopherol composition and/or level) can be modulated as outlined above.

Methods for assaying for a modulation in tocol content, tocopherol content and/or tocotrienol content are known in the art. For example, the total tocol content, tocopherol content and/or tocotrienol content of the seed or grain can be measured. Alternatively, the embryo tocol content, the tocopherol content and/or tocotrienol content of a seed or grain can be measured. Representative methods to measure tocol content, such as, extraction, immunopurification, chromatographic separation, gas chromatography-mass spectrometry, and quantification by ELISA methods can be found in, for example, Kamal-Eldi et al. (2000) J Chromatogr A 881:217-227; Bonvehi et al. (2000) J. AOAC Intl. 83:627-634; Goffman et al. (2001) J Agric. Food Chem. 49:4990-4994; Abidi (2000) J Chromatogr A 881:197-216; Gomez-Coronado et al. (2003) J. Agric Food Chem 51:5196-201; Panfili et al. (2003) J Agric Food Chem 51:3940-4; Huo et al. (1999) J Chromatogr B Biomed Sci Appl 724:249-55; U.S. Application Publication 20020042527; U.S. Pat. No. 5,908,940; U.S. Application Publication 2004/0034886; and, Frega et al. (1998) J. Amer. Oil Chem. Soc. 75:1723-1728. Each of these references is herein incorporated by reference. See also Examples 1 and 2 below.

Methods to assay for the activity of tocols are also known. For example, lipophilic antioxidant activity of tocols may be measured by various assays including the inhibition of the coupled auto-oxidation of linoleic acid and β-carotene and oxygen radical absorbance capacity (ORAC). See, Serbinova et al. (1994) Meth. Enzymol. 234:354-366; Emmons et al. (1999) J. Agric. Food Chem. 47:4894-4898); and, Huang et al. (2002) J. Agric. Food Chem. 52:2993-7. Such methods typically involve measuring the ability of antioxidant compounds (i.e., tocols) in test materials to inhibit the decline of fluorescence of a model substrate (fluorescein, phycoerythrin) induced by a peroxyl radical generator (2′,2′-azobis[20amidinopropane]dihydrochloride). See also, Andarwulan et al. (1999) J. Agric Food Chem 47:3158-63 and Fukuzawa et al. (1982) Lipids 17:511-3.

In specific embodiments, the compositions and methods of the invention modulate tocol content in a plant or plant part by modulating the level of a polypeptide having a LEC1-type B domain in combination with modulating the level of at least one other polypeptide involved in tocol biosynthesis. Alternatively, a CKC-like transcription factor can be employed in the methods and compositions of the invention. As demonstrated herein, modulating the level of a polypeptide having LEC1 activity and modulating the level of polypeptide involved in tocol biosynthesis results in a synergistic increase in tocol content in the plant or plant part.

### II. Compositions

A. LEC1-Like Polynucleotides and Polypeptides

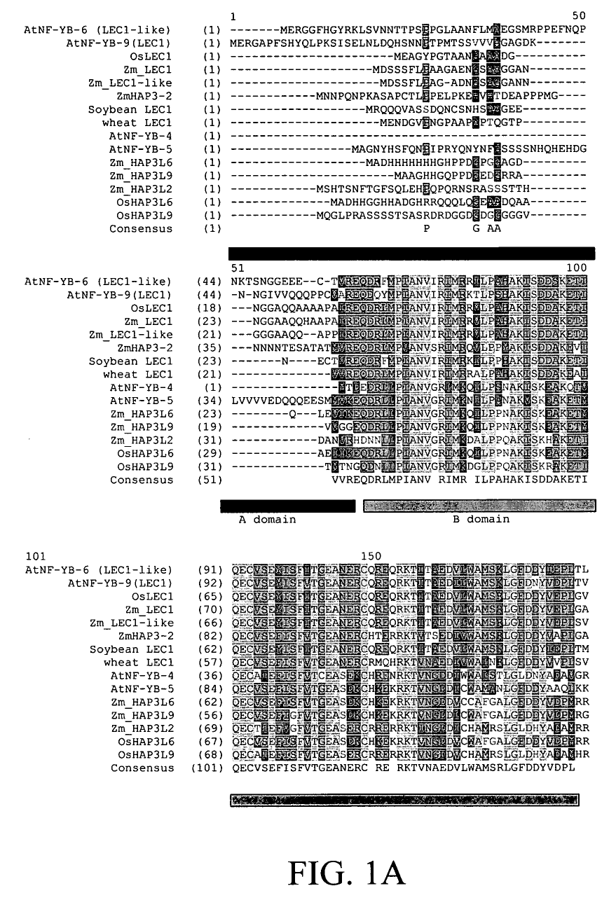

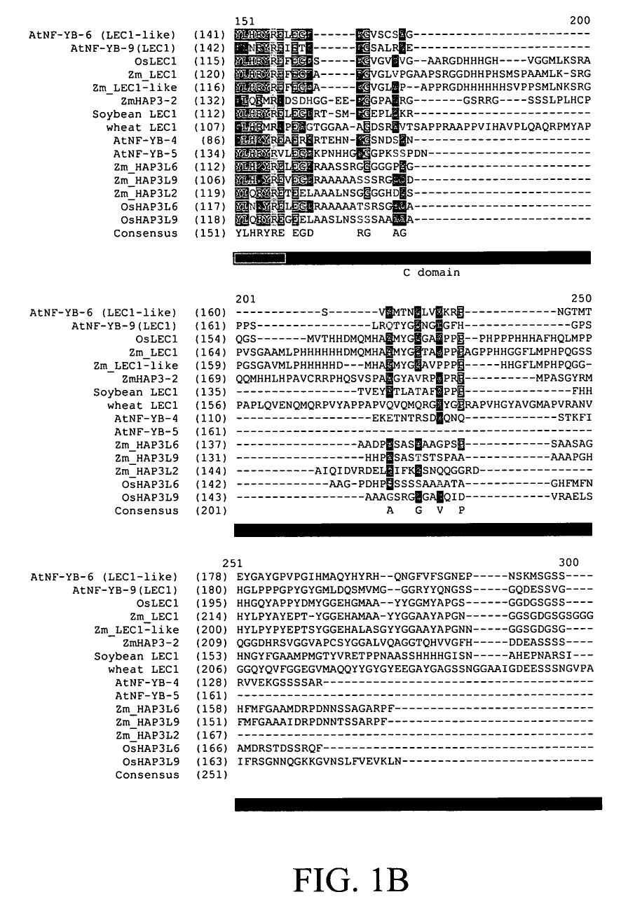

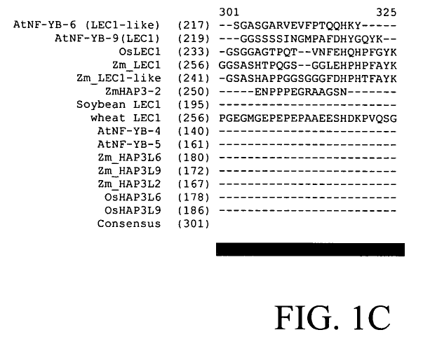

The Leafy Cotyledon 1 transcriptional activator (LEC1) is a member of the HAP (heme-activated protein)₃ transcription activator family whose members are characterized as having three regions: the A, B, and C domains. The central B domain is conserved among family members and comprises the conserved DNA binding CCAAT-box binding motif. FIG. 1 provides a sequence alignment of various members of the HAP3 transcriptional activator family and denotes the positions of domain A, B, and C and further shows the conserved CCAAT-box. Based on both sequence identity and function, members of the HAP3 family have been divided into two classes: members having a LEC1-type B domain and members having a non-LEC1-type B domain.

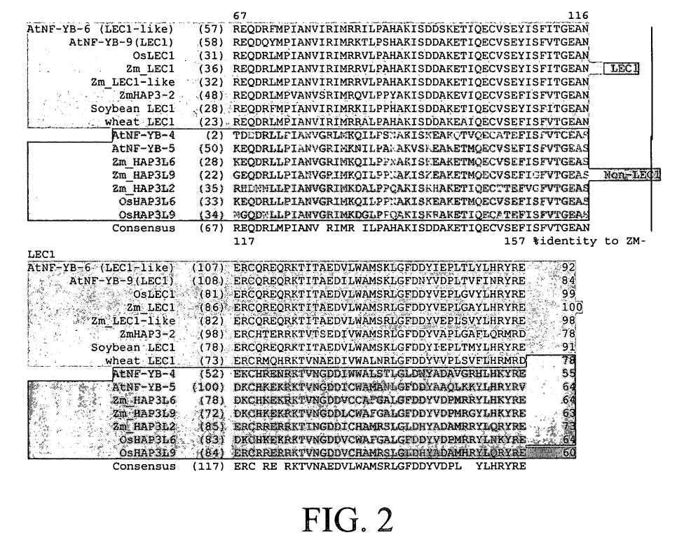

The B domains from various members of the HAP3 transcriptional activator family are aligned in FIG. 2. The B-domain for the Arabidopsis LEC1, from amino acid residue 28 to residue 117, shares between 55% and 63% identity (75-85% similarity) to other members of the HAP3 family, including maize (HAP3), chicken, lamprey, Xenopus, human, mouse, Emericella nidulens, Schizosaccharomyces pombe, Saccharomyces cerevisiae and Kluyveromyces lactis (Lotan et al. (1998) Cell 93: 1193-1205). The top, lightly shaded sequences are representative members of the LEC1-type B domain, while the bottom, darkly shaded sequences are representative of members of the non-LEC1-type domain.

Generally, the LEC1-type B domain comprises 16 conserved residues which are indicated by asterisks in FIG. 2. These 16 residues represent a consensus sequence (set forth in SEQ ID NO:3) for a LEC1-type B domain. It is recognized, however, that the 16 residues set forth in the consensus sequence for a LEC1-type B domain can be altered and still retain LEC1 activity. See, for example, Lee et al. (2003) PNAS 100:2152-2156, herein incorporated by reference in its entirety, which demonstrates specific alterations in some of the 16 conserved residues continues to allow the polypeptide to retain LEC1 activity. Amino acid R28 of SEQ ID NO:3 was found to play an important role in retaining LEC1 activity. In one embodiment, a LEC1-type B domain comprises the LEC-1 type B domain set forth in SEQ ID NO: 1 and 2 from the maize LEC1 polypeptide (SEQ ID NO: 4 and 5). Various polynucleotides and polypeptides having LEC1-type B-domains are set forth in US Publication Nos. 2003/0126638 and 2003/0204870, both of which are herein incorporated by reference.

As outlined in detail elsewhere herein, biologically active variants and fragments of the LEC1-type B domain can also be employed in the methods of the invention. Such variants and fragments are known in the art. See, for example, FIG. 2 and also Lee et al. (2003) PNAS 2152-2156 and U.S. Application Publication 2005/0034193.

Biologically active fragments and variants of a LEC1-type B domain will continue to retain LEC1 activity when the domain is placed within the context of a functional A and/or a functional C domain of a HAP3 transcriptional activator. As used herein, “LEC1 activity” is defined as the ability of a polypeptide to modulate tocol content in a plant or plant part. Methods are described above for assaying for an alteration in tocol content.

In one embodiment, the LEC1 polynucleotide or polypeptide employed in the invention comprises the polypeptide or polynucleotide set forth in SEQ ID NO: 4 and 5. As outlined in detail elsewhere herein, biologically active variants and fragments of the LEC1 polynucleotide and polypeptides can also be employed in the methods of the invention. Such variants and fragments are known in the art. See, for example, FIGS. 1 and 2 and also Lee et al. (2003) PNAS 2152-2156; Kwong et al. (2003) The Plant Cell 15:5-18; U.S. Patent Publication 2003/0126638; WO 02/57439, U.S. Pat. No. 6,825,397; U.S. Pat. No. 6,781,035; U.S. Application Publication 2005/0034193; and WO 98/37184, each of which is herein incorporated by reference.

As used herein, a “HAP3 transcriptional activator” comprises a member of the HAP3 family. This family of transcriptional activators is structurally well characterized. See, Li et al. (1992) Nucleic Acid Research 20:1087-1091; Xing et al. (1993) EMBO J. 12:4647-4655; Kim et al. (1996) Mol. Cell Biol. 16:4003-4013; Sinha et al. (1996) Mol Cell Biol 16:328-337; and, Lotan et al. (1998) Cell 93:1195-1205, each of which is herein incorporated by reference. In the methods and compositions of the invention, the HAP3 transcriptional activator comprises a LEC1-type B domain. Accordingly, a HAP3 transcriptional activator employed in the present invention can comprise a chimeric polypeptide having a functional A and/or a functional B domain from HAP3 transcriptional activator which in their native form may or may not have a LEC1-type B domain. See, for example, Lee et al. (2003) PNAS 2152-2156.

In another embodiment, the compositions and methods of the invention modulate tocol content in a plant or plant part by modulating the level of a CKC-like transcription factor in combination with modulating the level of at least one other polypeptide involved in tocol biosynthesis. Such CKC-like transcription factors and biologically active variant and fragments thereof that retain CKC-like activity are disclosed in, for example, U.S. Application Publication No. US2003/0204870, herein incorporated by reference in its entirety.

B. Tocol Biosynthesis Sequences

In one embodiment, the compositions and methods of the invention modulate tocol content by modulating the level of both a HAP3 transcriptional activator and at least one polypeptide involved in tocol biosynthesis. As used herein, a “polypeptide involved in tocol biosynthesis” comprises any polypeptide which is involved, either directly or indirectly, in modulating tocol content in a plant. Various methods to determine if a polypeptide is involved in tocol biosynthesis are discussed elsewhere herein. Such polypeptides include, but are not limited to, γ-tocopherol methyltransferase (U.S. Pat. No. 6,642,434, WO 99/04622, and Shintani et al. (1998) Science 282:2098-2100); p-hydroxyphenylpyruvate dioxygenase (HPPDase) (WO 97/27285, Garcia et al. (1997) Biochem J. 325:761 and Norris et al. (1998) Plant Physiol. 117:1317); tocopherol cyclase (U.S. Pat. No. 6,872,815 and Kanwisher et al. (2005) Plant Physiol. 137:713-723); 1-deoxy-D-xylose-5-phosphate synthase (WO 00/08169); geranylgeranyl-pyrophosphate oxidoreductase (WO 00/08169); tyrosine amino transferase (US Application Publication 20040086989); cyclase sxd1; geranylgeranyl reductase (GGR) (WO 99/23231); homogentisate geranylgeranyl transferases (HGGT); and, homogentisate phytyltransferase (HPT). Additional tocol biosynthetic polypeptides include polypeptides in the MEP pathway which increase the levels of tocopherol substrates such as isopentyl diphosphate (IPP) and dimethylallyl diphosphate (DMAPP) biosynthesis. Such polypeptides include ygbA, ygbP, ychB, yfgA. See, for example, U.S. Pat. No. 6,841,717.

In one embodiment, the polypeptide involved in tocol biosynthesis comprises a gamma-tocopherol methyltransferase sequences derived, for example, from cotton, maize, or the cyanobacteria Anabaena or a biologically active variant or fragment thereof. These sequences show similarity to gamma-tocopherol methyltransferase genes from Arabidopsis (PCT Publication No. WO 99/04622) and soybean (PCT Publication No. WO 00/032757). The heterologously expressed enzyme from maize, a monocotyledonous plant, showed an almost equal activity with tocopherol and tocotrienol substrates. On the other hand, gamma-tocopherol methyltransferase orthologs from the dicotyledenous plant cotton or blue-green algae showed only trace activities with tocotrienol substrates. In one embodiment, the methyltransferase is expressed in combination with an HGT polypeptide, a HGGT polypeptide or a biologically active variant or fragment thereof.

i. Homogentisate Geranylgeranyl Transferase (HGGT)

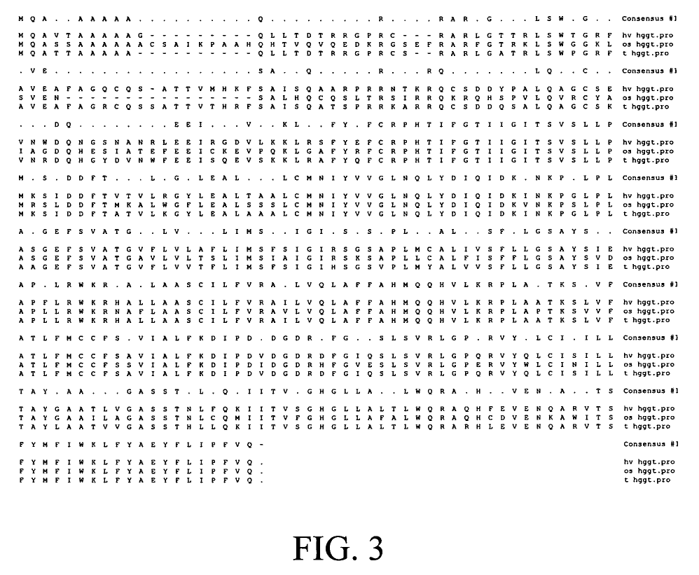

The family of homogentisate geranylgeranyl transferases (HGGT) comprises polypeptides that catalyze the condensation of homogentisate (or homogentisic acid) and geranylgeranyl pyrophosphate (or geranylgeranyl diphosphate). This reaction is an important step in tocotrienol biosynthesis and can result in the modulation of the tocol content. FIG. 3 provides a sequence alignment of various members of the HGGT family.

HGGT polypeptides are members of the UbiA prenyltransferase family. Members of this family are distinguished by the presence of a UbiA consensus motif. Of the known members of this family, HGGTs are most closely related to HPTs. Using amino acid sequence alignments, one skilled in the art can distinguish HGGT polypeptides from HPT polypeptides, other members of the UbiA prenyltransferase family. Amino acid residues that are conserved in HGGTs include (using SEQ ID NO: 7 as the basis for amino acid numbering): arginine 72, glutamine 73, cysteine 85, cysteine 118, phenylalanine 124, isoleucine 127, isoleucine 128, glycine 129, threonine 131, proline 137, aspartate 142, phenylalanine 144, threonine 145, cysteine 161, isoleucine 213, methionine 270, glutamine 272, leucine 279, alanine 280, isoleucine 333, threonine 338, threonine 351, glutamine 355, glycine 364, leucine 365, asparagine 381 and phenylalanine 401. It is recognized that each of these amino acids need not be present for a sequence to have HGGT activity.

HGGT polypeptides also are characterized as having specific protein motifs. Using the barley HGGT amino acid sequence as the basis for numbering (SEQ ID NO:7), HGGT-specific motifs include “FXXIIGXT” (SEQ ID NO: 10) which encompasses amino acids 124 through 131 and “(K/R)XXXDXFT” (SEQ ID NO: 11) which encompasses amino acids 139 through 145.

In one embodiment, the HGGT polynucleotide or polypeptide comprises the sequence set forth in SEQ ID NO: 6 and 7. As outlined in detail elsewhere herein, biologically active variants and fragments of the HGGT polynucleotide and polypeptide can also be employed in the methods of the invention. Such variants and fragments are known in the art. See, for example, U.S. Application Publication 2004/0034886, which is herein incorporated by reference.

Biologically active fragments and variants of a HGGT polypeptide will continue to retain HGGT activity. As used herein, “HGGT activity” is defined as the ability of a polypeptide to catalyzes the condensation of homogentisate (or homogentisic acid) and geranylgeranyl pyrophosphate (or geranylgeranyl diphosphate). Various methods are known in the art to assay for this activity. For example, the HGGT polypeptide can be expressed in a dicot which does not produce tocotrienols. Such a system includes tobacco callus which is enriched in tocopherol. Expression of an HGGT polypeptide in this system will increase accumulation of tocotrienols compared to the appropriate control plant or plant part. See, for example, Cahoon et al. (2003) Nature Biotechnology 21:1082-1087, and, US Application Publication 2004/0034886, herein incorporated by reference.

ii. Homogentisate Phytyltransferase (HPT)

The family of homogentisate phytyltransferase (HPT) polypeptides comprises polypeptides that catalyzes the condensation of homogentisate (or homogentisic acid) and phytyl pyrophosphate (or phytyl diphosphate). This reaction is believed to be the commitment step in tocopherol biosynthesis. See, for example, Cahoon et al. (2003) Nature Biotechnology 21:1082-1086. Other names that have been used to refer to this enzyme class include homogentisate phytyl pyrophosphate prenyltransferase, homogentisate phytyl diphosphate prenyltransferase, and phytyl/prenyl transferase.

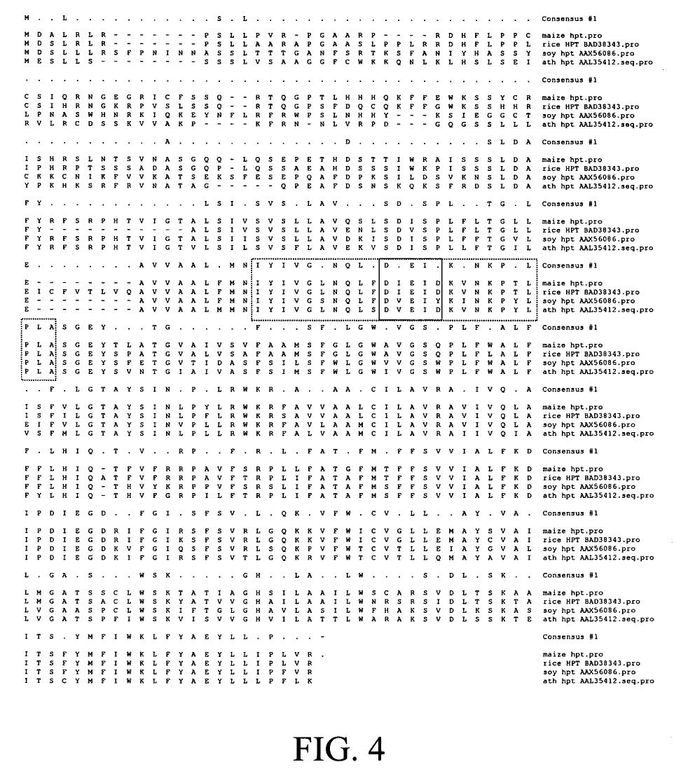

FIG. 4 provides a sequence alignment of various members of the HPT family. In one embodiment, the HPT polynucleotide or polypeptide comprises the sequence set forth in SEQ ID NO: 8 and 9. As outlined in detail elsewhere herein, biologically active variants and fragments of the HPT polynucleotide and polypeptides can also be employed in the methods of the invention. Such variants and fragments are known in the art. See, for example, the HPT sequences from Synechocystis sp. PCC 6803 HPT (GenBank Acc No. S74813), Rice HPT (GenBank Acc No. AX046728), soybean HPT (GenBank Acc No. AX046734), wheat HPT (GenBank Acc No. BE471221); Arabidopsis (GenBank AF324344); maize (GenBank Acc. No. AX046716); Collakova et al. (2001) Plant Physiology 127:1113-1124, Savidge et al. (2002) Plant Physiol. 129:321-332; Schledz et al. (2001) FEBS Lett. 499:15-20; and U.S. Pat. No. 6,787,683; each of which is herein incorporated by reference.

Biologically active fragments and variants of a HPT polypeptide will continue to retain HPT activity. In one method, a phytyl/prenyltransferase assay can be used to measure for HPT activity. For example, a HPT polypeptide can be expressed in a bacterium, such as E. coli, which lacks any enzymatic activity connected to tocol biosynthesis. HPT activity will be shown by an in vitro phytyl/prenyltransferase assay using protein extracts from E. coli expressing the polypeptide or by reconstruction of multiple steps of the pathway in E. coli. ¹⁴C uniformly labeled p-hydroxyphenyl pyruvate and phytyl-PP, or other prenyl diphosphates can be used as substrates. p-hydroxyphenyl pyruvate dioxygenase catalyses conversion of p-hydroxyphenylpyruvic acid to homogentisic acid, the immediate substrate for the tocopherol and plastoquinone prenyltransferase(s). Therefore, A. thaliana p-hydroxyphenylpyruvic acid dioxygenase (Norris et al. (1998) Plant Physiol. 117:1317) expressed in E. coli along with the prenyltransferase will be present in the reactions to couple the two enzymatic steps. To further determine HPT activity, the HPT polypeptide and wild type Synechocystis will be grown in the presence of ¹⁴C uniformly labeled L-tyrosine to trace prenylated products by using TLC and autoradiography.

In other assays for HPT activity, the HPT polypeptide can be expressed in E. coli in the presence of p-hydroxyphenylpyruvic acid dioxygenase (Norris et al. (1998) Plant Phys. 117:1317-1323), Adonis paleastina geranylgeranyl diphosphate synthase, and geranylgeranyl hydrogenase from Synechocystis (SLL1091, Addlesee et al. (1996) FEBS Lett. 389:126-130; Keller et al. (1998) Eur J Biochem 251:413-7). This allows for the reconstitution of the phytyl pyrophosphate pathway since E. coli does not possess any of these enzymatic activities. Lipids can be extracted and subjected to HPLC analysis as described in U.S. Pat. No. 6,787,683. If HPT activity is present, 2-methyl-6-phytylplastoquinone is stable and should be present in E. coli lipid extracts. See, also, Lopez et al. (1996) J. Bacteriol. 178:3369-3373 and Soll et al. (1980) Arch. Biochem. Biophys. 204:544-550 and Soll et al. (1984) Method Enzymol. 148:383-392.

C. Variants and Fragments

Fragments and variants of the polynucleotide encoding the polypeptide having the LEC1-type B domain, tocol biosynthesis polypeptides (i.e., HPT polypeptides or HGGT polypeptides or a methyltransferase), and proteins encoded thereby are also encompassed by the present invention. By “fragment” is intended a portion of the polynucleotide or a portion of the amino acid sequence and hence protein encoded thereby. Fragments of a polynucleotide may encode protein fragments that retain the biological activity of the native protein and hence have the biologically activities outlined elsewhere herein.

A fragment of a polynucleotide that encodes a biologically active portion of a polypeptide having a LEC1-type B domain, a LEC1-type B domain, or a tocol biosynthesis polypeptide (i.e., a HPT polypeptide or a HGGT polypeptide) will encode at least 15, 25, 30, 50, 75, 100, 125, 150, 175, 200, 225, or 250, 300, 350, 400, or more contiguous amino acids, or up to the total number of amino acids present in a full-length LEC1-type B domain, HAP3 transcriptional activator having a LEC1-type B domain, or a tocol biosynthesis polypeptide.

Thus, a fragment of a polynucleotide encoding a LEC1-type B domain, a HAP3 transcriptional activator having a LEC1-type B domain, or a tocol biosynthesis polypeptide (i.e., a HPT polypeptide or a HGGT polypeptide) may encode a biologically active portion of the protein. A biologically active portion of the protein can be prepared by isolating a portion of the polynucleotide, expressing the encoded portion of the protein (e.g., by recombinant expression in vitro), and assessing the activity of the encoded portion of the polypeptide. Polynucleotides that are fragments of a polynucleotide encoding a HAP3 transcriptional activator having a LEC1-type B domain or a tocol biosynthesis polypeptide (i.e., a HPT polypeptide or a HGGT polypeptide) comprise at least 16, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length polynucleotide disclosed herein.

“Variants” is intended to mean substantially similar sequences. For polynucleotide, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode a polypeptide useful in present invention Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 2, 3, 5, 7, and 9 can be employed in the methods and compositions of the invention. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, as described elsewhere herein. Such variants may result from, for example, genetic polymorphism or from human manipulation. Biologically active variants of a native protein employed in the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins employed in the methods and compositions of the invention may be altered in various ways including amino acid substitutions, deletions, truncations, and insertions. Methods for such manipulations are generally known in the art. For example, amino acid sequence variants and fragments of the proteins employed in the invention can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired activity, as discussed elsewhere herein. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by employing the assays discussed elsewhere herein.

Variant polynucleotides and proteins also encompass sequences and proteins derived from a mutagenic and recombinogenic procedure such as DNA shuffling. With such a procedure, one or more different sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a polynucleotide employed in the invention and other known genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The polynucleotides of the invention can be used to isolate corresponding sequences from other organisms, particularly other plants, more particularly other monocots. In this manner, methods such as PCR, hybridization, and the like can be used to identify such sequences based on their sequence homology to the sequence[s] set forth herein. Sequences isolated based on their sequence identity to the entire sequences of interest set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode for a LEC1-type B domain, a HAP3 transcriptional activator having a LEC1-type B domain or a tocol biosynthesis polypeptide (i.e., a HPT polypeptide or a HGGT polypeptide) and which hybridize under stringent conditions to the sequences disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant or organism of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among polynucleotide sequences and are optimally at least about 10 nucleotides in length, and most optimally at least about 20 nucleotides in length. Such probes may be used to amplify corresponding polynucleotide from a chosen plant or other organism by PCR. This technique may be used to isolate additional coding sequences from a desired plant or other organism or as a diagnostic assay to determine the presence of coding sequences in a plant or other an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 110° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 110° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

D. Plants, Plant Parts, Seeds, Grain and Oil

The invention provides plants, plant cells, and plant parts having altered tocol levels. In specific embodiments, a polypeptide comprising a LEC1-type B domain and an altered level of at least one polypeptide that is involved in tocol biosynthesis. In some embodiments, the plants of the invention have stably incorporated into their genome a heterologous polynucleotide encoding a polypeptide having a LEC1-type B domain and have at least one heterologous polynucleotide which is capable of modulating tocol biosynthesis. In specific embodiments, the polypeptide encoding the LEC1-type B-domain comprises a HAP3 transcriptional activator, such as, the sequence set forth in SEQ ID NO: 4 or a biologically active variant or fragment thereof, or the polynucleotide encodes the polypeptide of SEQ ID NO:5 or a biologically active variant or fragment thereof. Tocol biosynthesis polypeptides include, but are not limited to, HGGT, HPT, gamma-tocopherol methyltransferase, and biologically active variants or fragments thereof. In other embodiments, the plants of the invention comprise a polypeptide comprising a CKC-like transcription factor and an altered level of at least one polypeptide that is involved in tocol biosynthesis.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced or heterologous polynucleotides disclosed herein.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include plants that produce cereal grains (i.e., barley, maize, millet, oats, rye, rice sorghum, triticale, and wheat), oil-seed plants (i.e., canola, cotton, linseed, rapeseed, safflower, soybean, sunflower, Brassica, maize, alfalfa, palm, coconut,), and pulses (i.e., leguminous plants, such as, beans and peas). Beans include guar, locust bean, fenugreek, soybean, lupins, peanuts, garden beans, cowpea, mungbean, lima bean, fava bean, lentils, chickpea, etc.)

A “subject plant or plant cell” is one in which an alteration, such as transformation or introduction of a polypeptide, has occurred, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

Further provided are transgenic seed and/or transgenic grain having a modulated tocol content, a modulated tocotrienol content, and/or a modulated tocopherol content. For example, the seeds and grains of the invention can have a modulated tocol content. The total tocol content of the seed and/or the tocol content of the embryo of the seed can be at least, but not limited to, 100, 200, 300, 400, 500, 540, 600, 700, 800, 900, 1000, 1100, 1200, 1300, 1400, 1500, 1600, 1700, 1800, 1900, 2000 ppm, or greater or, alternatively, between about 150 to about 250, about 200 to about 350, about 300 to about 450, about 400 to about 550, about 500 to about 650, about 550 to about 700, about 600 to about 750, about 650 to about 800, about 750 to about 900, about 800 to about 950, about 850 to about 1000, about 900 to about 1050, about 950 to about 1200, about 1000 to about 1150, about 1100 to about 1250, about 1200 to about 1350 ppm or greater. In other embodiments, the total tocol content of the seed and/or the total tocol content of the embryo of the seed is about 1, 2, 3, 4, 5, 6, 7, 8, 9, 10, 11, 12, 13, 14, 15, 16, 17, 18, 19 or 20 times greater than the content found in an appropriate control.

In specific embodiments, transgenic maize seed and/or transgenic maize grain having a modulated tocol content, a modulated tocotrienol content, and/or a modulated tocopherol content are provided. In specific embodiments, the total tocol content of the maize seed and/or the tocol content of the embryo of the maize seed can be at least, but not limited to, 100, 200, 300, 400, 450, 460, 470, 480, 490, 500, 510, 525, 540, 550, 560, 575, 580, 600, 625, 650, 675, 700, 725, 750, 775, 800, 825, 830, 850, 900, 925, 950, 975, 1000, 1100, 1200, 1300, 1400, 1500, 1600, 1700, 1800, 1900, 2000 ppm greater. Alternatively, the total tocol content of the maize seed and/or the tocol content of the embryo of the maize seed can be at least, but not limited to, between about 400 to about 550, about 540 to about 1200, about 550 to about 900, about 600 to about 1000, about 650 to about 800, about 750 to about 900, about 800 to about 950, about 850 to about 1000, about 900 to about 1050, about 950 to about 1200, about 1000 to about 1150, about 1100 to about 1250, about 1200 to about 1350 ppm or greater.

In other embodiments, the seeds and grains of the invention can also have a modulated tocopherol content. For example, the total tocopherol content of the seed and/or the tocopherol content of the embryo of the seed can be at least, but not limited to, 100, 200, 300, 400, 500, 600, 700, 800, 900, 1000, 1100, 1200, 1300, 1400, 1500, 1600, 1700, 1800, 1900, 2000 ppm, or greater or between about 150 to about 250, about 200 to about 350, about 300 to about 450, about 400 to about 550, about 500 to about 650, about 550 to about 700, about 600 to about 750, about 650 to about 800, about 750 to about 900, about 800 to about 950, about 850 to about 1000, about 900 to about 1050, about 950 to about 1200, about 1000 to about 1150, about 1100 to about 1250, about 1200 to about 1350 ppm or greater. In other embodiments, the total tocopherol content of the seed and/or the total tocopherol content of the embryo of the seed is about 1, 2, 3, 4, 5, 6, 7, 8, 9, 10, 11, 12, 13, 14, 15, 16, 17, 18, 19 or 20 times greater than the content found in an appropriate control.

In other embodiments, the seeds and/or grains of the invention can also have a modulated tocotrienol content. For example, the total tocotrienol content of the seed and/or the tocotrienol content of the embryo can be at least, but not limited to, 100, 200, 300, 400, 460, 470, 480, 490, 500, 550, 575, 600, 700, 800, 900, 1000, 1100, 1200, 1300, 1400, 1500, 1600, 1700, 1800, 1900, 2000 ppm, or greater or between about 150 to about 250, about 200 to about 350, about 240 to about 790, about 300 to about 450, about 400 to about 550, about 500 to about 650, about 550 to about 700, about 600 to about 750, about 650 to about 800, about 750 to about 900, about 800 to about 950, about 850 to about 1000, about 900 to about 1050, about 950 to about 1200, about 1000 to about 1150, about 1100 to about 1250, about 1200 to about 1350, about 460 to about 2000 ppm or greater. In other embodiments, the total tocotrienol content of the seed and/or the total tocotrienol content of the embryo of the seed is about 1, 2, 3, 4, 5, 6, 7, 8, 9, 10, 11, 12, 13, 14, 15, 16, 17, 18, 19 or 20 times greater than the content found in an appropriate control.

In specific embodiments, the total tocotrienol content of the maize seed and/or the tocotrienol content of the embryo of the maize seed can be at least, but not limited to, 450, 460, 470, 480, 490, 500, 510, 525, 550, 560, 575, 580, 600, 625, 650, 675, 700, 725, 750, 775, 800, 825, 850, 900, 925, 950, 975, 1000, 1100, 1200, 1300, 1400, 1500, 1600, 1700, 1800, 1900, 2000 ppm greater. Alternatively, the total tocol content of the maize seed and/or the tocol content of the embryo of the maize seed can be at least, but not limited to, between about 460 to about 790, about 540 to about 1200, about 550 to about 900, about 600 to about 1000, about 650 to about 800, about 750 to about 900, about 800 to about 950, about 850 to about 1000, about 900 to about 1050, about 950 to about 1200, about 1000 to about 1150, about 1100 to about 1250, about 1200 to about 1350 ppm or greater.

Further provided are transgenic seed and/or transgenic grain having a modulated ratio of tocotrienol to tocopherol. The ratio of tocotrienol to tocopherol of the seed can be at least, but not limited to, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 35:1, 40:1, 50:1 or greater. In specific embodiment, the transgenic seed and/or transgenic grain having the modulate ratio of tocotrienol to tocopherol is from maize.

While any means can be used to produce the transgenic seed or grain having the increased tocol content, in one embodiment, the transgenic seed or grain comprises a heterologous polynucleotide encoding a polypeptide comprising a LEC1-type B-domain or the biologically active variant or fragment thereof and a heterologous polynucleotide which modulated level of at least one heterologous tocol biosynthesis polypeptide. Both of these sequences are operably linked to promoters that are active in the plant or plant part.

Further provided is a plant oil having an elevated tocol content, tocopherol content, and/or tocotrienol content. The oil can comprise a tocol, tocopherol content, and/or tocotrienol content of content of about 500, 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 18000, 19000, 20000, 21000, 22000, 23000, 24000, 25000 ppm or greater.

Method and compositions are further provided which allow for the conversion of the gamma tocol produced in the plant to alpha tocol. Compositions having the elevated levels of alpha tocol find use in many applications including, for example, increasing meat quality. In one method, the gamma tocol is converted into alpha tocol via the presence of a methyltransferase or a biologically active variant or fragment thereof. See, for example, U.S. Publication No. 2003154513, herein incorporated by reference. The methyltransferase can be provided to the plant via any method. In one embodiment, a polynucleotide encoding the methyltransferase is stably incorporated into the genome of the plant. In another embodiment, the polypeptide encoding the LEC-1 type B domain and the polynucleotide encoding the methyltransferase are stacked. Methods for stacking sequences are discussed in further detail elsewhere herein.

D. Polynucleotide Constructs

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotides employed in the invention can be provided in expression cassettes for expression in the plant of interest. The cassette will include 5′ and 3′ regulatory sequences operably linked to a polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a polynucleotide of the invention, and a transcriptional and translational termination region (i.e., termination region) functional in plants. The regulatory regions (i.e., promoters, transcriptional regulatory regions, and translational termination regions) and/or the polynucleotide of the invention may be native/analogous to the host cell or to each other. Alternatively, the regulatory regions and/or the polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of the protein in the plant or plant cell. Thus, the phenotype of the plant or plant cell can be altered.

The termination region may be native with the transcriptional initiation region, may be native with the operably linked polynucleotide of interest, may be native with the plant host, or may be derived from another source (i.e., foreign or heterologous) to the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In one embodiment, the polynucleotide employed in the invention is targeted to the chloroplast for expression. In this manner, where the polynucleotide of interest is not directly inserted into the chloroplast, the expression cassette will additionally contain a nucleic acid encoding a transit peptide to direct the gene product of interest to the chloroplasts. Such transit peptides are known in the art. See, for example, the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. The polynucleotides of interest to be targeted to the chloroplast may be optimized for expression in the chloroplast to account for differences in codon usage between the plant nucleus and this organelle. In this manner, the polynucleotide of interest may be synthesized using chloroplast-preferred codons. See, for example, U.S. Pat. No. 5,380,831, herein incorporated by reference.

In addition, the polypeptides employed in the invention can be targeted to other specific compartments within the plant cell. Methods for targeting polypeptides to a specific compartment are known in the art. Generally, such methods involve modifying the nucleotide sequence encoding the polypeptide in such a manner as to add or remove specific amino acids from the polypeptide encoded thereby. Such amino acids comprise targeting signals for targeting the polypeptide to a specific compartment such as, for example, a plastid, the nucleus, the endoplasmic reticulum, the vacuole, the mitochondrion, the peroxisome, the Golgi apparatus, and for secretion from the cell. Targeting sequences for targeting a polypeptide to a specific cellular compartment, or for secretion, are known to those of ordinary skill in the art.

A number of promoters can be used in the practice of the invention, including the native promoter of the polynucleotide sequence of interest. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a polynucleotide within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); mi1ps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference. The oleosin promoter and the Lpt2 promoters (for example, U.S. Pat. No. 6,013,862, WO95/15389 and WO 95/23230) can also be used.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

In certain embodiments, the polynucleotides employed in the invention can be stacked to create plants with a desired trait. A trait, as used herein, refers to the phenotype derived from a particular sequence or groups of sequences. For example, polynucleotide encoding a HAP3 transcriptional activator comprising a LEC1-type B domain may be stacked with one or more polynucleotides encoding a tocol biosynthesis polypeptide including, but not limited to, HPT and/or HGGT or biologically active variants or fragments thereof and/or a methyl transferase or a biologically active variant thereof.

These stacked combinations can be created by any method including, but not limited to, cross-breeding plants by any conventional or TopCross methodology, or genetic transformation. If the sequences are stacked by genetically transforming the plants, the polynucleotide sequences can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

F. Methods of Introducing

The methods of the invention modulate the level of a polypeptide. Such methods can be achieved by introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptide gains access to the interior of at least one cell of the plant. Methods for introducing a polynucleotide or a polypeptide into a plant are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the sequences employed in the invention can be provided to a plant using a variety of transient transformation methods. Such transient transformation methods include, but are not limited to, the introduction of a protein or variants and fragments thereof directly into the plant or the introduction of the a transcript encoding the protein into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, a polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. It is recognized that the polypeptides employed in the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

### III. Methods of Use

A. Methods for Modulating Tocol Content in a Plant or Plant Part

A method for modulating the level of a polypeptide comprising a LEC1-type B domain or a functional variant or fragment thereof in a plant in combination with modulating the level of at least one tocol biosynthesis polypeptide (i.e., a HPT polypeptide and/or a HGGT polypeptide or biologically active fragments or variants thereof) is provided.

A “modulated level” or “modulating level” of a polypeptide in the context of the methods of the present invention refers to any increase or decrease in the expression, concentration, or activity of a gene product, including any relative increment in expression, concentration or activity. Any method or composition that modulates expression of a target gene product, either at the level of transcription or translation, or modulates the activity of the target gene product can be used to achieve modulated expression, concentration, activity of the target gene product. In general, the level is increased or decreased by at least 1%, 5%, 10%, 20%, 30%, 40%, 50%, 60%, 70%, 80%, 90% or greater relative to a native control plant, plant part, or cell. Modulation in the present invention may occur during and/or subsequent to growth of the plant to the desired stage of development. In specific embodiments, the polypeptides of the present invention are modulated in monocots, particularly maize.

The level of the polypeptide having a LEC1-type B domain or a tocol biosynthesis polypeptide may be measured directly, for example, by assaying for the concentration of the polypeptide in the plant, or indirectly, for example, by measuring the amount of activity of the polypeptide in the plant. Methods for determining the activity of these polypeptides are described elsewhere herein.

In specific embodiments, the polypeptide or the polynucleotide of the invention is introduced into the plant cell. Subsequently, a plant cell having the introduced sequence of the invention is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to modulate the level of the targeted polypeptide in the plant. Plant forming conditions are well known in the art and discussed briefly elsewhere herein.

It is also recognized that the level of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,984; all of which are herein incorporated by reference. See also, WO 98/49350; WO 99/07865; WO 99/25821; and, Beetham et al. (1999) Proc. Natl. Acad. Sci. USA 96:8774-8778; herein incorporated by reference.

It is therefore recognized that methods of the present invention do not depend on the incorporation of the entire polynucleotide into the genome, only that the plant or cell thereof is altered as a result of the introduction of a polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprises at least one nucleotide.

In one embodiment, the level of the polynucleotide encoding a polypeptide having a LEC1-type B domain is increased in combination with an increase in the level of a tocol biosynthesis polypeptide (i.e., a HPT polypeptide or a HGGT polypeptide or a biologically active variant or fragment thereof). An increase in the level of these polypeptides can be achieved by providing to the plant a polynucleotide encoding a polypeptide having a LEC1-type B domain or a biologically active variant or fragment thereof and providing to the plant a polynucleotide that is capable of modulating the level of a tocol biosynthesis polypeptide (i.e., nucleotide sequence encoding an HPT polypeptide or HGGT polypeptide, or a biologically active variant or fragment thereof). As discussed elsewhere herein, many methods are known in the art for providing a polypeptide to a plant including, but not limited to, direct introduction of the polypeptide into the plant, introducing into the plant (transiently or stably) a polynucleotide construct encoding a polypeptide having the appropriate activity as described elsewhere herein. It is also recognized that the methods of the invention may employ a polynucleotide that is not capable of directing, in the transformed plant, the expression of a protein or an RNA. Thus, the level of a polypeptide having a LEC1-type B domain or a tocol biosynthesis polypeptide may be increased by altering the gene encoding the respective polypeptide or its promoter. See, e.g., Kmiec, U.S. Pat. No. 5,565,350; Zarling et al., PCT/US93/03868. Therefore, mutagenized plants that carry mutations in a polynucleotide encoding a polypeptide having a LEC1-type B domain and/or a tocol biosynthesis polypeptide where the mutations increase expression of the polynucleotide encoding a polypeptide having a LEC1-type B domain and/or a tocol biosynthesis polypeptide are provided.

In other embodiments, the level of the polynucleotide encoding a polypeptide having a LEC1-type B domain and a tocol biosynthesis polypeptide (i.e., a HPT polypeptide, or a HGGT polypeptide or a biologically active variant or fragment thereof) is reduced or eliminated by introducing into a plant a polynucleotide that inhibits the level of the polypeptide of the invention. The polynucleotide may inhibit the expression of the polypeptide directly, by preventing translation of the appropriate messenger RNA, or indirectly, by encoding a polypeptide that inhibits the transcription or translation of a polynucleotide having a LEC1-type B domain and/or a tocol biosynthesis polypeptide. Methods for inhibiting or eliminating the expression of a gene in a plant are well known in the art, and any such method may be used in the present invention to inhibit the expression of the desired polynucleotide in a plant. In other embodiments of the invention, the activity of a polynucleotide encoding a polypeptide having a LEC1-type B-domain is reduced or eliminated by transforming a plant cell with a sequence encoding a polypeptide that inhibits the activity of the polypeptide of interest. In other embodiments, the activity of a polynucleotide encoding a polypeptide having a LEC1-type B domain and/or a tocol biosynthesis polypeptide may be reduced or eliminated by disrupting the gene encoding the polypeptide. The invention encompasses mutagenized plants that carry mutations in polynucleotides encoding a polypeptide having a LEC1-type B domain and/or a tocol biosynthesis polypeptide, where the mutations reduce expression of the gene or inhibit the activity of the encoded polypeptide.

Reduction of the activity of specific genes (also known as gene silencing or gene suppression) is desirable for several aspects of genetic engineering in plants. Many techniques for gene silencing are well known to one of skill in the art, including, but not limited to, antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; WO 98/53083; Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, U.S. Patent Publication No. 20030175965; Panstruga et al. (2003) Mol. Biol. Rep. 30:135-140; Wesley et al. (2001) Plant J. 27:581-590; Wang and Waterhouse (2001) Curr. Opin. Plant Biol. 5:146-150; U.S. Patent Publication No. 20030180945; and, WO 02/00904, all of which are herein incorporated by reference); ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); each of which is herein incorporated by reference; and other methods or combinations of the above methods known to those of skill in the art.

It is recognized that with the polynucleotides of the invention, antisense constructions, complementary to at least a portion of the messenger RNA (mRNA) for the polynucleotide encoding a polypeptide having a LEC1-type B domain and/or a tocol biosynthesis polypeptide can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used.

The polynucleotides of the present invention may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference. Thus, many methods may be used to reduce or eliminate the activity of a polypeptide having a LEC1-type B-domain and/or a tocol biosynthesis polypeptide. More than one method may be used to reduce the activity of a single polypeptide. In addition, combinations of methods may be employed to reduce or eliminate the activity of the polypeptides.

II. Methods to Improve Feed Quality, Shelf-Life and Oil Preparations

The antioxidant capacity of tocols contribute to the nutritive value of food products and animal feeds. Tocol supplementation of various livestock feed products, such as, swine, beef, and poultry feeds, has been shown to significantly improve tissue quality and extend the shelf-life of post-processed meat products by retarding post-processing lipid oxidation, which contributes to undesirable flavor components. Improved tissue quality can be reflected in, for example, improved color score, reduced purge, increased shelf life, higher pH, greater oxidative stability, and beneficial effects on sensory data, such as, freshness, tenderness, and juiciness. See, for example, Ball (1988) Fat-soluble vitamin assays in food analysis. A comprehensive review. London: Elsevier Science Publishers LTD; Sante et al. (1994) J. Sci. Food Agric. 65(4):503-507; Buckley et al. (1995) J. of Animal Science 73:3122-3130; Asghar et al. (1991) J. Sci. Food Agric. 57:31-41; Aalhaus et al. (2001) Advances in Pork Production 12:145-150; and, Cannon et al. (1995) J. of Animal Science 74:98-105, each of which is herein incorporated by reference.

Accordingly, methods are provided to improve the tissue quality of an animal by feeding an animal a diet having an elevated tocol content. Such methods comprise feeding the animal a diet comprising a sufficient amount of a grain or oil of the invention which comprises an elevated tocol content, tocotrienol content, and/or tocopherol content.

In one embodiment, such methods comprise feeding a diet comprising a sufficient amount of a grain where the grain comprises a polynucleotide encoding a polypeptide having the LEC1-type B-domain or biologically active variant or fragment thereof and at least one polynucleotide that modulates the level of a polypeptide involved in tocol biosynthesis. In specific embodiments, the polynucleotide that can modulate tocol biosynthesis encodes a HGGT polypeptide and/or a HPT polypeptide or a biologically active fragment or variant thereof.

The feed employed in the diet can comprise about 5%, 10%, 20%, 30%, 40%, 50%, 60%, 70%, 80%, 90%, or 100% of the grain having the elevated tocol content. In specific embodiments, the grain comprises a polynucleotide encoding a polypeptide having the LEC1-type B-domain or biologically active variant or fragment thereof and at least one polynucleotide that modulates the level of a polypeptide involved in tocol biosynthesis. In other embodiments, the feed employed in the diet can comprise about 1 to about 15%, about 10 to about 25%, about 20 to about 35%, about 30 to about 45%, about 40% to about 55%, about 50 to about 65%, about 60 to about 75%, about 70 to about 85%, about 80% to about 95% or about 90% to 100% of the grain with the elevated tocol content.

In specific embodiments, the feed in the diet comprises a grain of the invention (in specific embodiments a maize grain) and the diet comprises about 60% to about 85%, about 65% to about 75%, or about 70% to about 75% of the grain. Alternatively, diet can comprise about 60%, 65%, 70%, 75%, 80%, 85%, 90% or greater of grain having the elevated tocol content.

The total tocol content, tocopherol content and/or tocotrienol content of the feed employed in the methods of the invention will be sufficient to improve the tissue quality of the animal consuming the feed. In specific embodiments, the feed supplied to the animals is formulated to comprises a total level of tocol, tocopherol and/or tocotrienol of about 50, 60, 75, 100, 125, 150, 160, 170, 180, 190, 200, 225, 250, 275, 300, 325, 350, 375, 400, 425, 450, 475, 500, 525, 550, 575, 600, 625, 650, 700, 800 ppm or greater. In other embodiments, the feed supplied to the animals is formulated to comprises a total level of tocol, tocopherol and/or tocotrienol of about 150 to about 700 ppm, about 180 to about 620 ppm, or about 175 to about 550 ppm.

The amount of tocol, tocotrienol, and tocopherol supplied in the grain or the oil of the diet will be an amount sufficient to improve the tissue quality of the animal. For example, the amount of tocol, tocotrienol, and tocopherol in the diet will result in a concentration of tocol, tocotrienol, and tocopherol in the animal tissue of about 0.8 mg/kg tissue or about 1 mg/kg tissue. In other embodiments, the amount of tocol, tocotrienol, and/or tocopherol supplied in the grain or the oil of the diet will be an amount sufficient to result in the concentration of tocol, tocotrienol, and/or tocopherol in the tissue of the animal to be 0.5, 1, 2, 4, 5, 6, 7, 8, 9, 10, 11, 12, 13, 14 time or greater than the level found in an appropriate animal control not consuming the diet.

The diet can be supplied for any number of days such that allows the tissue quality of the animal to improve. Accordingly, in specific embodiments, the diet if feed for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 weeks or longer.

The tissue quality of any animal can be improved. Animals of interest include, but are not limited to, ruminant animals, including, but not limited to, cattle, bison, or lamb, as well as, non-ruminant animals including, but not limited to, swine, poultry (i.e., chickens, layer hens, turkey, ostriches and emu) or fish.

Feed quality, and ultimately tissue quality, can further be improved by including in the diet the increased tocol content in combination with a high oil content and/or a high oleic acid content. The oleic acid may be in the form of vegetable oil having an elevated level of oleic acid, including, but not limited to: high-oleic corn, sunflower, soybean, cotton, cocoa, peanut, safflower, or canola oil. The oleic acid may also be fed in the form of these oilseed or grain crops arising from plants genetically modified to express a high-oleic trait. The genetically modified oilseed or grain may be modified by transgenic methods well known in the art, or as naturally occurring or induced mutations. A “high oleic” trait is a trait wherein the oilseed or grain exhibits a greater than wild-type level of oleic fatty acid. See WO Pub. 94/11516, WO Pub. 90/10380, WO Pub. 91/11906, and U.S. Pat. No. 4,627,192 herein incorporated in their entirety by reference. A fat or oil source with an iodine value comparable to or lower than a high-oleic vegetable oil source such as, but not limited to, high-oleic sunflower oil may also be utilized.

In one embodiment, the diet fed to improve animal tissue quality comprises feeding a grain having an increased tocol content and also having a high oil content and/or a high oleic acid content. The high oleic acid content in the grain can be naturally occurring, or alternatively, they can be modified by human intervention. Methods of increasing oleic acid level are known in the art, as are methods to increase oil content. See, for example, U.S. Pat. Nos. 6,372,965, 6,737,564; 6,483,008; 6,388,113; 6,169,190 and PCT/US96/09486. Each of the references is herein incorporated by reference. In one embodiment, the oleic acid and the selected tocols are added to the feed in the form of a corn grain arising on ears of corn plants that express the FAD-2 in combination with a heterologous polynucleotide encoding a polypeptide having a LEC1-type B domain an at least one heterologous polynucleotide that modulates the level of a tocol biosynthesis polypeptide. Accordingly, the present invention provides plants and plant parts having an increased tocol content and a high oil content and/or a high oleic acid content.

Increased tocol levels in plants are also associated with enhanced stability and extended shelf-life of fresh and processed plant products. See, for example, Peterson (1995) Cereal-Chem 72(1):21-24; Ball (1988) Fat-soluble vitamin assays infood analysis. A comprehensive review. London: Elsevier Science Publishers LTD. Accordingly, methods are provided to improving the nutritive value and shelf-life of food products and animal feeds. Such methods comprise increasing the level of a HAP3 transcriptional activator having a LEC1-type B domain in a plant or plant part and thereby increasing tocol content of the plant or plant part. The nutritive value and shelf-life of food products and animal feeds can also be improved by increasing the level of the HAP3 transcriptional activator having a LEC1-type B domain in combination with modulating the level of at least one other polypeptide involved in tocol biosynthesis. In specific embodiments, the nutritive value and shelf-life of food products and animal feeds is improved by increasing the level of the polypeptide having the LEC1-type B-domain in combination with increasing the level of a HGGT polypeptide and/or a HPT polypeptide or a biologically active fragment or variant thereof.

Tocols are extracted with vegetable oils during the commercial processing of oil seeds. Genetic enhancement of tocol levels in seeds can therefore be used as a method for producing vegetable oil with improved shelf-life and oxidative ability for cooking and industrial lubricant applications. In addition, oil having an increase in tocol content would also have an enhanced nutritional value. Accordingly, methods to produce vegetable oil having an increased tocol content are provided. Such methods comprise increasing the level of the polypeptide having a LEC1-type B domain in combination with modulating the level of at least one other polypeptide involved in tocol biosynthesis. In specific embodiments, the increased tocol content in the vegetable oil is achieved by increasing the level of the polypeptide having the LEC1-type B-domain in combination with increasing the level of a HGGT polypeptide and/or a HPT polypeptide or a biologically active fragment or variant thereof. Routine oil extraction methods are preformed on the plant or plant part to obtain the oil having the modulated tocol content.

It is further recognized that the plant or plant part produced by the methods of the invention can also be used to extract tocols from the plant product. Based on the diverse therapeutic properties of tocols, such plant extracts can be used as neutracuetical.

In specific embodiments, the plant, plant part, seed, grain, or oil is a constituent of animal feed or a food product. In another embodiment, the plant part having the modulated tocol content is a fruit, more preferably a fruit with an enhanced shelf-life. Plants or parts thereof of the present invention can be utilized in methods, for example without limitation, to obtain a seed, meal, feedstock, or oil with a modulated level of tocol content. Plants utilized in such methods may be processed. Accordingly, the present invention provides seed, grain, feed, and/or oil that are produced from a plant or plant part having a modulated tocol content as described herein. Methods to produce feed, meal, protein and oil preparations from various plant parts are known in the art. See, for example, U.S. Pat. Nos. 4,957,748, 5,100,679, 5,219,596, 5,936,069, 6,005,076, 6,146,669, and 6,156,227. Further provided is meat produced from animals being feed the plant, plant part, grain and/or oil having a modulated tocol content, as described elsewhere herein.

IV. Methods to Improve Plant Resistance to Oxidative Stress

Tocols affects plant health much as it does human health, i.e., by scavenging free radicals, thus protecting plant membrane integrity. Plants exhibiting high levels of antioxidants have greater resistance to oxidative damage. See, Harper et al. (1978) Plant Cell. Environ. 1:211-215; Dhindsa et al. (1981) J. Exp. Bot. 32:79-91; Wise et al. (1987) Plant Physiol. 83:278-282; Monk et al. (1989) Physiol. Plant. 75:411-416; and, Spychalla et al. (1990) Plant Physiol. 94:1214-1218. Accordingly, methods are also provided to increase the resistance of a plant or plant part to oxidative stress. Oxidative stress refers to any condition that results in the formation of active oxygen species that damage a plant. Oxidative stress can arise from high irradiance, drought, heat, or salinity. This improved resistance may result in an improved productivity of crop plants (i.e., under stress of drought and heat) and an increased storage life of seeds and horticultural plant material.

The method for increasing resistance to oxidative stresses comprises increasing the level of the polypeptide having a LEC1-type B domain in combination with modulating the level of at least one other polypeptide involved in tocol biosynthesis. In specific embodiments, the improved resistance to oxidative stress can is achieved by increasing the level of the polypeptide having the LEC1-type B-domain in combination with increasing the level of a HGGT polypeptide and/or a HPT polypeptide or a biologically active fragment or variant thereof.

The following examples are offered by way of illustration and not by way of limitation.

### EXPERIMENTAL

### Example 1

### Overexpression of Maize LEC1 Increases Tocopherol Content in Maize Kernel

### Vector Construction

Maize LEC1 was moved into an expression cassette containing a barley LTP2 promoter which expressed in aleurone and embryo (WO 95/15389 and WO 95/23230) and a PinII terminator (An et al. (1989) Plant Cell 1:115-122). This cassette was then subcloned adjacent to a Ubiquitin promoter:Mo-PAT expression cassette. The resulting expression cassettes flanked by T-DNA border sequences were then introduced into the Agrobacterium “super-binary” vector using electroporation.

### Agrobacterium-Mediated Maize Transformation

For Agrobacterium-mediated transformation of maize with the Lec1 construct described above, the method of Zhao was employed (U.S. Pat. No. 5,981,840, and PCT patent publication WO98/32326; the contents of which are hereby incorporated by reference). Briefly, immature embryos were isolated from maize and the embryos contacted with a suspension of Agrobacterium, where the bacteria are capable of transferring the Lec1 construct to at least one cell of at least one of the immature embryos (step 1: the infection step). In this step the immature embryos were immersed in an Agrobacterium suspension for the initiation of inoculation. The embryos were co-cultured for a time with the Agrobacterium (step 2: the co-cultivation step). The immature embryos were cultured on solid medium following the infection step. Following this co-cultivation period an optional “resting” step is contemplated. In this resting step, the embryos were incubated in the presence of at least one antibiotic known to inhibit the growth of Agrobacterium without the addition of a selective agent for plant transformants (step 3: resting step). The immature embryos were cultured on solid medium with antibiotic, but without a selecting agent, for elimination of Agrobacterium and for a resting phase for the infected cells. Next, inoculated embryos were cultured on medium containing a selective agent and growing transformed callus were recovered (step 4: the selection step). The immature embryos were cultured on solid medium with a selective agent resulting in the selective growth of transformed cells. The callus was then regenerated into plants (step 5: the regeneration step), and calli grown on selective medium are cultured on solid medium to regenerate the plants.

### Determine Effect of LEC1 Overexpression on Tocopherol Pathway Gene Expression

Tocopherol content in the LEC1 transgenic plants was determined using the procedures outline in detail in Example 2. Gene expression profiling by Lynx was performed as described (Brenner et al. (2000) Proc Natl Acad Sci USA 97:1165-70). Immature embryos were dissected from transgenic and null kernels at 25 days after pollination. Expression of tocopherol pathway genes in LEC1 embryo was compared to null embryo to determine if LEC1 affect gene expression from tocopherol pathway.

### Results

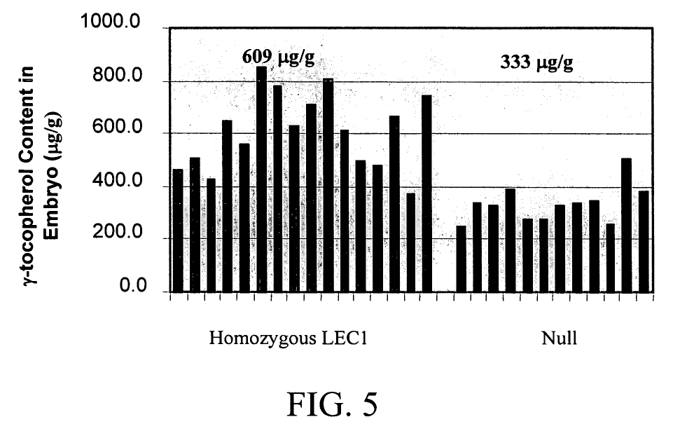

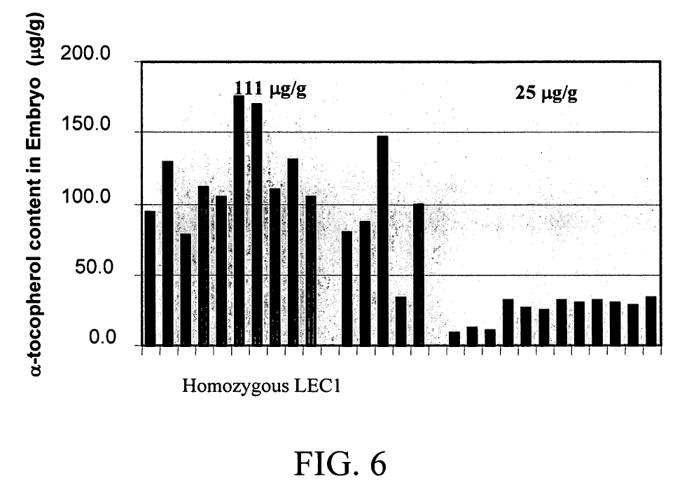

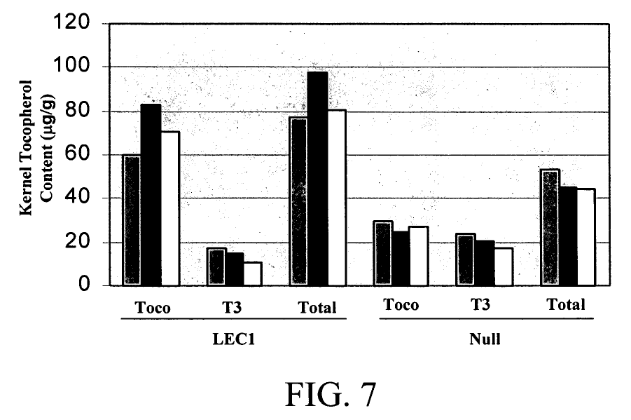

We have analyzed embryo tocopherol content in 16 homozygous LEC1 T2 ears from two transgenic events and 12 corresponding null ears. LEC1 embryo contains 609 μg γ-tocopherol per g embryo in average while null embryo contains 333 μg γ-tocopherol per g embryo. LEC1 increases embryo γ-tocopherol content by 83% (FIG. 5). α-tocopherol even showed a more significant increase in LEC1 embryo. α-tocopherol content in LEC1 embryo is 4-fold higher than α-tocopherol content in null embryo (FIG. 6). We then measured total tocopherol content in whole grain meal. LEC1 kernels contain 85.3 μg total toco per g grain which is much higher than 47.7 μg/g total toco content in null grain (FIG. 7).

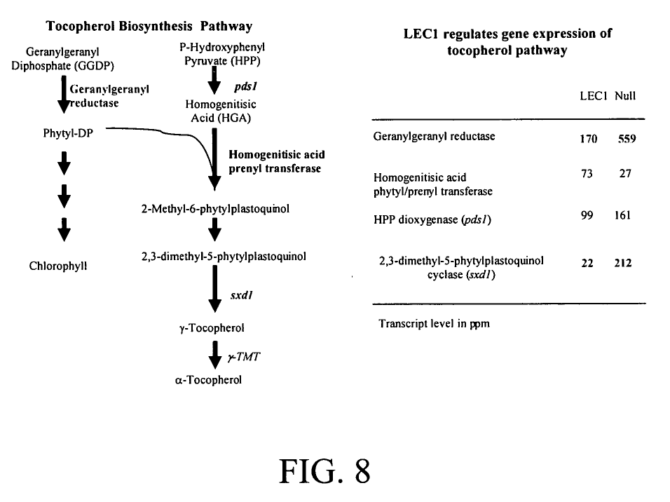

To understand how LEC1 increases tocopherol content, we determined if LEC1 regulates gene expression in tocopherol biosynthesis pathway. Lynx profiling data indicated that geranylgeranyl reductase and Sdx1 gene are up-regulated 3-fold and 10-fold respectively in LEC1 embryo, whereas prenyl transferase gene (HPT) is down-regulated 2-3 fold and Pds1 gene expression is not changed. See, FIG. 8. The results suggested that LEC1 increased grain tocopherol content by regulating gene expression of tocopherol biosynthesis pathway.

### Example 2

### Effect of zmLEC1 and HGGT on Tocopherol and Tocotrienol Content

To study the effect that LEC1 exerts on tocopherol and tocotrienol content in transgenic lines overexpressing HGGT, oil, tocopherol and tocotrienol content of transgenic corn events generated with PHP20884 and PHP20941 were examined. The two constructs are identical with each containing a Fad2 hairpin under the control of the oleosin promoter and barley HGGT under control of EAP1 promoter with the exception that PHP20941 also contains the LTP2 LEC1 expression cassette. The DNA constructs PHP20884 and PHP20941 were generated as follows. The HV-HGGT coding sequence was PCR-modified to generate a BsaI site (with an NcoI-compatible overhang) at the start codon and a SmaI site just after the stop codon. This sequence-confirmed fragment was then ligated into a cloning intermediate containing the EAP1 PRO and EAP1 TERM (see attached sequence information). The first intron from the maize ADH1 gene was inserted as an EcoRV/SmaI fragment at the unique Eco47III site between the promoter and the coding sequence. The final HGGT expression cassette plasmid was designated PHP20752. For PHP20884, this cassette was added to a Japan Tobacco binary already containing the OLE PRO: FAD2-1 inverted repeat construct (described in US Publication No. 2005/016494) as well as a selectable marker gene for bialaphos resistance. For PHP20941, the LTP2 PRO:ZM-LEC1:PINII TERM expression cassette (also described in US Publication No. 2005/016494) was ligated into PHP20752 just 5′ to the HV-HGGT expression cassette just described and both genes were moved as a single BstEII fragment into the Japan Tobacco binary vector already containing the OLE PRO:FAD2-1 inverted repeat and the selectable marker gene. These binary vectors were introduced (separately) into Agrobacterium tumefaciens LBA4404 containing pSB1 (aka PHP10523) by electroporation and the resulting cointegrate plasmids (PHP20884 and PHP20941) confirmed by restriction endonuclease digests. Transgenic corn plants were generated with PHP20884 and PHP20941 as described in Example 1.

431 and 501 events were generated and analyzed for PHP20884 and PHP20941, respectively. Quantitative analysis of tocopherols and tocotrienols of T1 kernels was conducted as follows. Briefly, ten T1 kernels of each event were ground were ground in a FOSS tecator sample mill (FOSS, USA) using a 1 mm screen. 300 mg of tissue were extracted in 1 ml of heptane for two hours; alpha-tocopherol acetate was added as internal standard at a final concentration of 38 μg ml⁻¹. 10 μL of filtered heptane extract was subjected to HPLC using a Lichrospher column (250-4 HPLC cartridge, Si60, 5 μM particle size) using heptane containing 1.5% isopropanol as mobile phase at a flow rate of 1 mL min⁻¹ on an Agilent 1100 HPLC system (Agilent, USA). External standards of α, γ and δ tocopherols and tocotrienols (2.5 μg mL⁻¹), separated under identical conditions were used for tocol quantitation. Tocols were detected using a fluorescence detector using excitation and emission wavelengths of 295 nm, 330 nm, respectively. Oil content of the heptane extract was determined as follows. 25 microliters of heptane extract were supplied with 5 microliters of heptadecanoic acid (10 mg mL⁻¹) as internal standard and 500 microliters of 1% sodium methoxide in methanol. Samples were incubated at 50° C. for 2 h. One mL of 1 M NaCl was added. Fatty acid methylesters were extracted into 1 mL of heptane. Four μL of heptane extract were analyzed on Hewlett-Packard 6890 Gas Chromatograph fitted with an Omegawax 320 fused silica capillary column (Supelco Inc., Cat#24152). The oven temperature was programmed to hold at 220 C for 2.7 min, increase to 240 C at 20 C/min and then hold for an additional 2.3 min. Carrier gas was supplied by a Whatman hydrogen generator. Retention times were compared to those for methyl esters of standards commercially available (Nu-Chek Prep, Inc. catalog #U-99-A).

TABLE 1
Synergistic effect of LEC1 and HGGT
on increasing tocotrienol content

| | | PHP20884 | PHP20941 | |
|---|---|---|---|---|
| | | (−Lec1) | (+Lec1) | |
| | | n = 431 | n = 501 | % increase |
| oil (%) | average | 2.4 | 3 | 25 |
| | high | 3.4 | 4.4 | 29 |
| tocopherol (ppm) | average | 31 | 41 | 32 |
| | high | 41 | 79 | 93 |
| tocotrienol (ppm) | average | 110 | 179 | 63 |
| | high | 271 | 352 | 30 |

TABLE 2
Synergistic effect of LEC1 and HGGT on tocotrienol
is independent of effect on kernel oil content

| | PHP20884 | PHP20941 | |
|---|---|---|---|
| | (−Lec1) | (+Lec1) | |
| | n = 206 | n = 254 | % increase |
| avg tocopherol | 34 | 37 | 9 |
| avg tocotrienol | 119 | 159 | 34 |

Above, table 1 shows that Lec1 expression increases kernel oil content by 25-30%. In agreement with experiments reported previously in Example 1 Lec1 also increases tocopherol content significantly: the average and highest observed tocopherol content of PHP20941 events is 32 and 92% higher, respectively than that of PHP20884 events. Lec1 also increases HGGT mediated tocotrienol production significantly: the average and highest observed tocotrienol content of PHP20941 events is 63 and 30% higher, respectively than that of PHP20884 events. Thus co-expression of LEC1 and HGGT in the embryo increases the threshold of tocotrienol accumulation by 30% when compared to lines that express only HGGT, even though LEC1 alone does not significantly affect tocotrienol accumulation (see Example 1) We also observed that in a subset of lines generated with PHP20941 in which oil biosynthesis was not elevated tocotrienol biosynthesis was still increased when compared to a subset of 20884 lines with similar oil levels (Table 2) Thus, the effect that Lec1 has on tocotrienol synthesis appears to be independent of the signaling mechanism that leads to elevated oil biosynthesis. In other words, at least some of the signaling components controlled by LEC1 that lead to increased tocotrienol biosynthesis must be different from those that lead to increased oil biosynthesis. Bulk measurements of oil and tocol content of T1 grain provide valuable initial information about transgene efficiency in metabolic engineering experiments. However, T1 grain derived from a selfed T0 plant in most cases still contains 25% of untransformed seed. Thus the data reported so far are derived from samples diluted with untransformed grain. Single seed from T2 seed of selected events were subjected to HPLC and GC analysis to get a more accurate picture of oil and tocol levels in PHP20941 transgenics. Briefly, T1 plants of several events generated with PHP20941 were either allowed to self or crossed to inbreds GR581, EE05F and ED705. Eight kernels were analyzed derived from individual ears of T2 plants. For outcross kernels, we expect that transgenic and null segregates at 1:1 ratio. Tocol and oil analysis was performed as described above. Kernels were pulverized using the Geno Grinder tissue homogenizer (Glen Mills, USA).

TABLE 3
Tocotrienol content in T2 segregating
LEC1/HGGT transgenic kernels

| | tocph. | toct. | tocochromanol | |
|---|---|---|---|---|
| Event ID | (ppm) | (ppm) | (ppm oil) | % oil |
| E6312.12.11.11 T2 EE05F | 46 | 415 | 11633 | 4.0 |
| | 37 | 395 | 11764 | 3.7 |
| | 32 | 328 | 12056 | 3.0 |
| | 31 | 303 | 10694 | 3.1 |
| | 43 | 27 | 2175 | 3.2 |
| | 49 | 16 | 1852 | 3.5 |
| | 46 | 13 | 1731 | 3.4 |
| | 29 | 9 | 1417 | 2.7 |
| E6312.12.9.28 T2 EE05F | 38 | 527 | 16259 | 3.5 |
| | 39 | 466 | 15703 | 3.2 |
| | 34 | 430 | 16885 | 2.7 |
| | 34 | 382 | 15951 | 2.6 |
| | 13 | 27 | 1670 | 2.4 |
| | 34 | 15 | 1575 | 3.1 |
| | 17 | 12 | 1614 | 1.9 |
| | 15 | 8 | 1212 | 1.9 |
| E6312.12.9.28 T2 ED705 | 40 | 759 | 28612 | 2.8 |
| | 40 | 554 | 21111 | 2.8 |
| | 25 | 519 | 18364 | 3.0 |
| | 40 | 50 | 4264 | 2.1 |
| | 34 | 37 | 2776 | 2.5 |
| | 28 | 32 | 2901 | 2.1 |
| | 36 | 29 | 2489 | 2.6 |
| | 30 | 25 | 2326 | 2.4 |
| E6312.12.9.31 T2 ED705 | 41 | 790 | 20089 | 4.1 |
| | 26 | 647 | 19824 | 3.4 |
| | 64 | 329 | 12007 | 3.3 |
| | 65 | 39 | 3238 | 3.2 |
| | 62 | 33 | 3280 | 2.9 |
| | 64 | 32 | 3437 | 2.8 |
| | 47 | 31 | 2688 | 2.9 |
| | 51 | 30 | 2839 | 2.8 |
| E6312.12.9.6 T2 GR581 | 110 | 450 | 16194 | 3.5 |
| | 73 | 331 | 8868 | 4.6 |
| | 44 | 325 | 9350 | 3.9 |
| | 74 | 320 | 9610 | 4.1 |
| | 42 | 18 | 2590 | 2.3 |
| | 49 | 17 | 1886 | 3.5 |
| | 45 | 14 | 2350 | 2.5 |
| | 42 | 10 | 1661 | 3.1 |
| E6312.12.10.23 T2 SELF | 66 | 455 | 14839 | 3.5 |
| | 67 | 386 | 11328 | 4.0 |
| | 70 | 377 | 12101 | 3.7 |
| | 68 | 344 | 13187 | 3.1 |
| | 69 | 337 | 12798 | 3.2 |
| | 67 | 323 | 12605 | 3.1 |
| | 65 | 281 | 12019 | 2.9 |
| | 45 | 239 | 10286 | 2.8 |

Table 3 illustrates that co-expression of Lec1 and barley HGGT gene in the corn embryo leads to production of grain with a tocotrienol content as high as 790 ppm. Moreover, the oil that can be extracted for this grain has a tocotrienol content as high as 14000 ppm.

### Example 3

### Modulating the Nutritional Value of a Plant

Immature maize embryos from greenhouse donor plants are bombarded with a plasmid containing a polynucleotide encoding LEC1 operably linked to a Ltp2 promoter (WO 95/15389 and WO 95/2323) and the selectable marker gene PAT (Wohlleben et al. (1988) Gene 70:25-37), which confers resistance to the herbicide Bialaphos. The plasmid further comprises a Fad2 hairpin under the control of the oleosin promoter and barley HGGT under control of EAP1 promoter. Alternatively, the selectable marker gene is provided on a separate plasmid. Transformation is performed as follows. Media recipes follow below.

### Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the polynucleotide encoding the LEC1 polypeptide operably linked to the Ltp2 promoter and encoding the HGGT polypeptide under the control of the EAP1 promoter. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun #HE34-1 or #HE34-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for an increase in tocol content. Methods to assay for tocol content are described in detail in Examples 2 and 3.

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 4

### Methods of Modulate Tocol Content in Soybean

Soybean embryos are bombarded with a plasmid containing the LEC1 polynucleotide operably linked to a Ltp2 promoter and the barely HGGT polypeptide operably linked to the EAP1 promoter. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the LEC1 polynucleotide operably linked to the Ltp2 promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 5

### Improving Quality of Animal Tissue

A swine feeding trial is performed to determine the effects on meat quality of supplementation of a swine diet with corn grain arising on ears of corn plants that over express zmLEC1 and HGGT. See, Example 2.

Forty barrows (˜75 lbs.) are randomly placed into individual pens with water and feed provided ad libitum. The pigs receive a common diet for seven days. On the eighth day the pigs are weighed individually and a uniform group of 24 pigs with an average body weight of about 75 pounds is selected. The pigs are randomly assigned one of two dietary treatments with 12 pigs per treatment. The feeding trial is initiated for at least 3 months.

Both the control and the test group pigs are fed a corn-soybean meal diet formulated to provide adequate levels of all nutrients. In addition, the test group pigs are feed a dietary treatment comprising grain having stably incorporated into its genome a heterologous polynucleotide comprising zmLEC1 and HGGTm, wherein the grain constitutes about 70% to about 75% of the animal feed and the final tocol concentration of the feed is about 180 ppm to about 620 ppm.

The pigs are harvested. Following a twelve-hour feed withdrawal, the pigs are transported to a commercial processing facility. At the commercial processing facility, individual hot carcass weight, back fat depth and loin depth are measured and recorded on the day of slaughter. Loin pH, loin color value (Minolta L*), Marbling and fat firmness are recorded 24 hours post mortem.

Trimmed bellies are collected to measure the effects of dietary treatments on ground pork oxidation rate. The bellies are ground through a meat grinder and mixed. Four one-pound samples of each ground belly are placed on a retail meat tray and covered with oxygen permeable film. On each of days 1, 7, 10 and 16 post-grinding, one of the trays is opened and a sample submitted for TBARS (thiobarbituric acid reactive substances) determination, a measure of the extent of oxidation.

Approximately twenty-one days after slaughter the loins are removed from vacuum bags and weighed. The liquid that accumulates in the bags during storage is measured to calculate 21-day purge. Loin pH is measured at three locations by carefully inserting a glass probe into the mid-point of the anterior, mid and posterior thirds of each loin. Measurement location (blade, chop or shoulder) can affect loin pH, Hunter L, L* and a* values.

The Hunter L, a and b values are measured with a Hunter laboratory system for color evaluation. Hunter L is a measurement of the lightness of an object, and may be thought of as the light reflectance from the surface of an object. Thus, a higher Hunter L value indicates a lighter color, and an L value of 100 would indicate prefect reflectance from the surface. An increasingly positive Hunter a value indicates a redder color, and an increasingly positive Hunter b value indicates a more yellow color.

A one-inch thick chop is collected from the 10^{th} rib region of each pig carcass to evaluate the effects of dietary treatment on cooked product characteristics. Sensory evaluation is conducted using a trained sensory panel. Each panelist evaluates a ½ inch cube removed from the center of a cooked pork chop immediately after reaching 71 degrees C. Samples are evaluated for degree of juiciness, tenderness, chewiness, pork flavor and off-flavor intensity.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for increasing tocol content in a plant or part thereof, comprising
a) introducing into the plant or the plant part a heterologous polynucleotide encoding a polypeptide comprising a LEC1-type B-domain having an amino acid sequence set forth in SEQ ID NO:2 or a biologically active variant or a fragment of SEQ ID NO:2, wherein said biologically active variant comprises at least 80% sequence identity to SEQ ID NO:2, said polypeptide or the biologically active variant or fragment thereof have LEC1 activity;
b) introducing into the plant or the plant part a heterologous polynucleotide which modulates the level of at least one tocol biosynthesis polypeptide; and,
thereby increasing the tocol content in said plant or said plant part.

2. The method of claim 1, wherein the heterologous polynucleotide encoding the polypeptide comprising the LEC1-type B-domain or the biologically active variant or fragment encodes a polypeptide thereof is selected from the group consisting of
a) a polypeptide comprising an amino acid sequence having at least 80% sequence identity to SEQ ID NO: 2 or 5, wherein said polypeptide has LEC1 activity;
b) a polypeptide comprising an amino acid sequence set forth as in SEQ ID NO:2, 3, or 5; and,
c) a polypeptide comprising at least 50 consecutive an amino acids of SEQ ID NO:2 or 5, wherein said polypeptide has LEC1 activity.

3. The method of claim 1, wherein said heterologous polynucleotide encoding the polypeptide comprising the LEC1-type B-domain or the biologically active variant thereof is selected from the group consisting of:
a) a polynucleotide comprising a nucleotide sequence set forth in SEQ ID NO:1 or 4;
b) a polynucleotide comprising a nucleotide sequence having at least 80% sequence identity to SEQ ID NO: 1 or 4, wherein said polynucleotide encodes a polypeptide having LEC1 activity;
c) a polynucleotide comprising at least 60 consecutive nucleotides of SEQ ID NO:1 or 4, wherein said polynucleotide encodes a polypeptide having LEC1 activity;
d) a polynucleotide encoding a polypeptide having at least 80% sequence identity to SEQ ID NO:2 or 5, wherein said polynucleotide encodes a polypeptide having LEC1 activity;
e) a polynucleotide encoding a polypeptide set forth in SEQ ID NO: 2, 3 or 5; and,
f) a polynucleotide encoding at least 50 consecutive amino acids of SEQ ID NO:2 or 5, wherein said polynucleotide encodes a polypeptide having LEC1 activity.

4. The method of claim 1, wherein said modulation comprises an increase in the level of at least one tocol biosynthesis polypeptide.

5. The method of claim 4, wherein said polynucleotide that modulates the level of the tocol biosynthesis polypeptide encodes a polypeptide selected from the group consisting of:
a) a polypeptide comprising an amino acid sequence set forth in SEQ ID NO:7;
b) a polypeptide comprising at least 80% sequence identity to SEQ ID NO:7, wherein said polypeptide comprises HGGT activity; and,
c) a polypeptide comprising at least 50 consecutive amino acids of SEQ ID NO:7, wherein said polypeptide comprises HGGT activity.

6. The method of claim 4, wherein said polynucleotide that modulates the level of the tocol biosynthesis polypeptide is selected from the group consisting of:
a) a polynucleotide comprising a nucleotide sequence set forth in SEQ ID NO:6;
b) a polynucleotide comprising a nucleotide sequence having at least 80% sequence identity to SEQ ID NO: 6, wherein said polynucleotide encodes a polypeptide having HGGT activity;
c) a polynucleotide comprising at least 60 consecutive nucleotides of SEQ ID NO:6, wherein said polynucleotide encodes a polypeptide having HGGT activity;
d) a polynucleotide encoding a polypeptide having at least 80% sequence identity to SEQ ID NO:7, wherein said polynucleotide encodes a polypeptide having HGGT activity;
e) a polynucleotide encoding a polypeptide set forth in SEQ ID NO: 7; and,
f) a polynucleotide encoding at least 50 consecutive amino acids of SEQ ID NO:7, wherein said polynucleotide encodes a polypeptide having HGGT activity.

7. The method of claim 1, wherein said increase in tocol content comprises an increase in tocotrienol content.

8. The method of claim 1, wherein said increase in tocol content comprises an increase in tocopherol content.

9. The method of claim 4, wherein said polynucleotide that modulates the level of the tocol biosynthesis polypeptide encodes a polypeptide selected from the group consisting of:
a) a polypeptide comprising the amino acid sequence set forth in SEQ ID NO:9;
b) a polypeptide having at least 80% sequence identity to the amino acid sequence set forth in SEQ ID NO:9, wherein said polypeptide has HPT activity; and,
c) a polypeptide comprising at least 50 consecutive amino acids of SEQ ID NO:9, wherein said polynucleotide has HPT activity.

10. The method of claim 4, wherein said polynucleotide that modulates the level of the tocol biosynthesis polypeptide is selected from the group consisting of:
a) a polynucleotide comprising a nucleotide sequence set forth in SEQ ID NO:8;
b) a polynucleotide comprising a nucleotide sequence having at least 80% sequence identity to SEQ ID NO: 8, wherein said polynucleotide encodes a polypeptide having HPT activity;
c) a polynucleotide comprising at least 60 consecutive nucleotides of SEQ ID NO:8, wherein said polynucleotide encodes a polypeptide having HPT activity;
d) a polynucleotide encoding a polypeptide having at least 80% sequence identity to SEQ ID NO:9, wherein said polynucleotide encodes a polypeptide having HPT activity; or,
e) a polynucleotide encoding a polypeptide set forth in SEQ ID NO: 9.

11. The method of claim 1, wherein said plant or plant part has a phenotype selected from the group consisting of:
a) an enhanced tolerance to oxidative stress; and,
b) an enhanced nutritional value.

12. The method of claim 1, wherein said plant is a dicot.

13. The method of claim 12, wherein said dicot is soybean, Brassica, sunflower, cotton, or alfalfa.

14. The method of claim 1, wherein said plant is a monocot.

15. The method of claim 14, wherein said monocot is maize, wheat, rice, barley, sorghum, or rye.

16. A plant or part thereof comprising
a) a heterologous polynucleotide encoding a polypeptide comprising a LEC1-type B-domain having the amino acid sequence set forth in SEQ ID NO:2 or a biologically active variant or a fragment thereof, wherein said biologically active variant comprises at least 60% sequence identity to SEQ ID NO:2, said polypeptide or the biologically active variant or fragment thereof having LEC1 activity operably linked to a first promoter active in the plant or plant part; and,
b) a heterologous polynucleotide which modulated level of at least one heterologous tocol biosynthesis polypeptide operably linked to a second promoter active in the plant or plant part.

17. The plant or plant part of claim 16, wherein the polynucleotide encoding the polypeptide comprising the LEC1-type B-domain or the biologically active variant or fragment thereof encodes a polypeptide selected from the group consisting of
a) a polypeptide comprising an amino acid sequence having at least 80% sequence identity to SEQ ID NO: 2 or 5, wherein said polypeptide has LEC1 activity;
b) a polypeptide comprising an amino acid sequence set forth as in SEQ ID NO:2, 3, or 5; and,
c) a polypeptide comprising at least 50 consecutive amino acids of SEQ ID NO:2 or 5, wherein said polypeptide has LEC1 activity.

18. The plant or plant part of claim 16, wherein said polynucleotide encoding the polypeptide comprising the LEC1-type B-domain or the biologically active variant or fragment thereof is selected from the group consisting of:
a) a polynucleotide comprising a nucleotide sequence set forth in SEQ ID NO:1 or 4;
b) a polynucleotide comprising a nucleotide sequence having at least 80% sequence identity to SEQ ID NO: 1 or 4, wherein said polynucleotide encodes a polypeptide having LEC1 activity;
c) a polynucleotide comprising at least 60 consecutive nucleotides of SEQ ID NO:4, wherein said polynucleotide encodes a polypeptide having LEC1 activity;
d) a polynucleotide encoding a polypeptide having at least 80% sequence identity to SEQ ID NO:2 or 5, wherein said polynucleotide encodes a polypeptide having LEC1 activity;
e) a polynucleotide encoding a polypeptide set forth in SEQ ID NO: 2, 3 or 5; and,
f) a polynucleotide encoding at least 50 consecutive amino acids of SEQ ID NO:2 or 5, wherein said polynucleotide encodes a polypeptide having LEC1 activity.

19. The plant or plant part of claim 16, wherein said modulated level of said tocol biosynthesis polypeptide comprises an increase of at least one heterologous tocol biosynthesis polypeptide.

20. The plant or plant part of claim 19, wherein said heterologous polynucleotide encoding said tocol biosynthesis polypeptide encodes a polypeptide selected from the group consisting of:
a) a polypeptide comprising an amino acid sequence set forth in SEQ ID NO:7;
b) a polypeptide having at least 80% sequence identity to SEQ ID NO:7, wherein said polypeptide comprises HGGT activity; and,
c) a polypeptide comprising at least 50 consecutive amino acids of SEQ ID NO:7, wherein said polypeptide comprises HGGT activity.

21. The plant or plant part of claim 19, wherein said polynucleotide encoding said tocol biosynthetic polypeptide is selected from the group consisting of:
a) a polynucleotide comprising a nucleotide sequence set forth in SEQ ID NO:6;
b) a polynucleotide comprising a nucleotide sequence having at least 80% sequence identity to SEQ ID NO: 6, wherein said polynucleotide encodes a polypeptide having HGGT activity;
c) a polynucleotide comprising at least 60 consecutive nucleotides of SEQ ID NO:6, wherein said polynucleotide encodes a polypeptide having HGGT activity;
d) a polynucleotide encoding a polypeptide having at least 80% sequence identity to SEQ ID NO:7, wherein said polynucleotide encodes a polypeptide having HGGT activity;
e) a polynucleotide encoding a polypeptide set forth in SEQ ID NO: 7; and,
f) a polynucleotide encoding at least 50 consecutive amino acids of SEQ ID NO:7, wherein said polynucleotide encodes a polypeptide having HGGT activity.

22. The plant or plant part of claim 16, wherein said increased tocol content comprises an increased tocotrienol content.

23. The plant or plant part of claim 16, wherein said increased tocol content comprises an increased tocopherol content.

24. The plant or plant part of claim 19, wherein said heterologous polynucleotide which modulates the level of a tocol biosynthesis polypeptide encodes a polypeptide selected from the group consisting of:
a) a polypeptide comprising the amino acid sequence set forth in SEQ ID NO:9;
b) a polypeptide having at least 80% sequence identity to SEQ ID NO:9, wherein said polypeptide has HPT activity; and,
c) a polypeptide comprising at least 50 consecutive amino acids of SEQ ID NO:9, wherein said polypeptide has HPT activity.

25. The plant or plant part of claim 19, wherein said polynucleotide which can modulate the level of a tocol biosynthesis polypeptide encodes a polypeptide selected from the group consisting of:
a) a polynucleotide comprising a nucleotide sequence set forth in SEQ ID NO:8;
b) a polynucleotide comprising a nucleotide sequence having at least 80% sequence identity to SEQ ID NO: 8, wherein said polynucleotide encodes a polypeptide having HPT activity;
c) a polynucleotide comprising at least 60 consecutive nucleotides of SEQ ID NO:8, wherein said polynucleotide encodes a polypeptide having HPT activity;
d) a polynucleotide encoding a polypeptide having at least 80% sequence identity to SEQ ID NO:9, wherein said polynucleotide encodes a polypeptide having HPT activity;
e) a polynucleotide encoding a polypeptide set forth in SEQ ID NO: 9; and,
f) a polynucleotide encoding at least 50 consecutive amino acids of SEQ ID NO:9, wherein said polynucleotide encodes a polypeptide having HPT activity.

26. The plant or plant part of claim 16, wherein said plant or plant part comprises a phenotype selected from the group consisting of:
a) an enhanced tolerance to oxidative stress; and,
b) an enhanced nutritional value.

27. The plant or plant part of claim 16, wherein said plant is a dicot.

28. The plant or plant part of claim 27, wherein said dicot is soybean, Brassica, sunflower, cotton, or alfalfa.

29. The plant or plant part of claim 16, wherein said plant or plant part is a monocot.

30. The plant or plant part of claim 29, wherein said monocot is maize, wheat, rice, barley, sorghum, or rye.

31. A transgenic seed from the plant of claim 16.

32. A transgenic grain produced from the plant of claim 16.

33. A maize transgenic seed or grain having an embryo tocotrienol content of at least 460 ppm.

34. A maize transgenic seed or grain having an embryo tocol content of at least 540 ppm.

35. A maize transgenic seed or grain having a tocotrienol to tocopherol ratio of about 11:1.


SEQUENCE LISTING (32 sequences)

SEQ ID NO: 1 - DNA, 273 bp, Zea mays
  CDS (1)...(273)
cgcgagcaggaccggctgatgccgatcgcgaacgtgatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgag

SEQ ID NO: 2 - protein, 91 aa, Zea mays
REQDRLMPIANVIRIMRRVLPAHAKISDDAKETIQECVSEYISFITGEANERCQREQRKTITAEDVLWAMSRLGFDDYVEPLGAYLHRYRE

SEQ ID NO: 3 - protein, 65 aa, Artificial Sequence
  consensus sequence for LEC-1 B domain
REQDXXMPIANVIRIMRXXLPXHAKISDDAKEXIQECVSEYISFXTXEANXRCXXXXRKTXXXEX

SEQ ID NO: 4 - DNA, 834 bp, Zea mays
  CDS (1)...(834)
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtgatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaag

SEQ ID NO: 5 - protein, 278 aa, Zea mays
MDSSSFLPAAGAENGSAAGGANNGGAAQQHAAPAIREQDRLMPIANVIRIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 6 - DNA, 1221 bp, Hordeum vulgare
  CDS (1)...(1221)
atgcaagccgtcacggcggcggccgcggcggggcagctgctaacagatacgaggagagggcccagatgtagggctcggctgggaacgacgagattatcctggacaggtcgatttgcagtggaagcttttgcaggccagtgccaaagtgctactactgtaatgcataaattcagtgccatttctcaagctgctaggcctagaagaaacacaaagagacagtgcagcgatgattatccagccctccaagctggatgcagcgaggttaattgggatcaaaacggttccaacgccaatcggcttgaggaaatcaggggagatgttttgaagaaattgcgctctttctatgaattttgcaggccacacacaatttttggcactataataggtataacttcagtgtctctcctgccaatgaagagcatagatgattttactgtcacggtactacgaggatatctcgaggctttgactgctgctttatgtatgaacatttatgtggtcgggctgaatcagctatatgacattcagattgacaagatcaacaagccaggtcttccattggcatctggggaattttcagtagcaactggagttttcttagtactcgcattcctgatcatgagctttagcataggaatacgttccggatcggcgccactgatgtgtgctttaattgtcagcttccttcttggaagtgcgtactccattgaggctccgttcctccggtggaaacggcacgcgctcctcgctgcatcatgtatcctatttgtgagggctatcttggtccagttggctttctttgcacatatgcagcaacatgttctgaaaaggccattggcagcaaccaaatcgctggtgtttgcaacattgtttatgtgttgcttctctgccgtcatagcactattcaaggatattccagatgttgatggagatcgagactttggtatccaatccttgagtgtgagattggggcctcaaagagtgtatcagctctgcataagcatattgttgacagcctatggcgctgccactctagtaggagcttcatccacaaacctatttcaaaagatcatcactgtgtctggtcatggcctgcttgctttgacactttggcagagagcgcagcactttgaggttgaaaaccaagcgcgtgtcacatcattttacatgttcatttggaagctattctatgcagagtatttccttataccatttgtgcag

SEQ ID NO: 7 - protein, 407 aa, Hordeum vulgare
MQAVTAAAAAGQLLTDTRRGPRCRARLGTTRLSWTGRFAVEAFAGQCQSATTVMHKFSAISQAARPRRNTKRQCSDDYPALQAGCSEVNWDQNGSNANRLEEIRGDVLKKLRSFYEFCRPHTIFGTIIGITSVSLLPMKSIDDFTVTVLRGYLEALTAALCMNIYVVGLNQLYDIQIDKINKPGLPLASGEFSVATGVFLVLAFLIMSFSIGIRSGSAPLMCALIVSFLLGSAYSIEAPFLRWKRHALLAASCILFVRAILVQLAFFAHMQQHVLKRPLAATKSLVFATLFMCCFSAVIALFKDIPDVDGDRDFGIQSLSVRLGPQRVYQLCISILLTAYGAATLVGASSTNLFQKIITVSGHGLLALTLWQRAQHFEVENQARVTSFYMFIWKLFYAEYFLIPFVQ

SEQ ID NO: 8 - DNA, 1197 bp, Zea mays
  CDS (1)...(1197)
atggacgcgcttcgcctacggccgtccctcctccccgtgcggcccggcgcggcccgcccgcgagatcattttctaccaccatgttgttccatacaacgaaatggtgaaggacgaatttgcttttctagccaaaggacccaaggtcctaccttgcatcaccatcagaaattcttcgaatggaaatcctcctattgtaggatatcacatcggtcattaaatacttctgttaatgcttcggggcaacagctgcagtctgaacctgaaacacatgattctacaaccatctggagggcaatatcatcttctctagatgcattttacagattttcccggccacatactgtcataggaacagcattaagcatagtctcagtttcccttctagctgtccagagcttgtctgatatatcacctttgttcctcactggtttgctggaggcagtggtagctgcccttttcatgaatatctatattgttggactgaaccagttattcgacattgagatagacaaggttaacaagccaactcttccattggcatctggggaatacacccttgcaactggggttgcaatagtttcggtctttgccgctatgagctttggccttggatgggctgttggatcacaacctctgttttgggctcttttcataagctttgttcttgggactgcatattcaatcaatctgccgtaccttcgatggaagagatttgctgttgttgcagcactgtgcatattagcagttcgtgcagtgattgttcagctggccttttttctccacattcagacttttgttttcaggagaccggcagtgttttctaggccattattatttgcaactggatttatgacgttcttctctgttgtaatagcactattcaaggatatacctgacatcgaaggggaccgcatattcgggatccgatccttcagcgtccggttagggcaaaagaaggtcttttggatctgcgttggcttgcttgagatggcctacagcgttgcgatactgatgggagctacctcttcctgtttgtggagcaaaacagcaaccatcgctggccattccatacttgccgcgatcctatggagctgcgcgcgatcggtggacttgacgagcaaagccgcaataacgtccttctacatgttcatctggaagctgttctacgcggagtacctgctcatccctctggtgcgg

SEQ ID NO: 9 - protein, 399 aa, Zea mays
MDALRLRPSLLPVRPGAARPRDHFLPPCCSIQRNGEGRICFSSQRTQGPTLHHHQKFFEWKSSYCRISHRSLNTSVNASGQQLQSEPETHDSTTIWRAISSSLDAFYRFSRPHTVIGTALSIVSVSLLAVQSLSDISPLFLTGLLEAVVAALFMNIYIVGLNQLFDIEIDKVNKPTLPLASGEYTLATGVAIVSVFAAMSFGLGWAVGSQPLFWALFISFVLGTAYSINLPYLRWKRFAVVAALCILAVRAVIVQLAFFLHIQTFVFRRPAVFSRPLLFATGFMTFFSVVIALFKDIPDIEGDRIFGIRSFSVRLGQKKVFWICVGLLEMAYSVAILMGATSSCLWSKTATIAGHSILAAILWSCARSVDLTSKAAITSFYMFIWKLFYAEYLLIPLVR

SEQ ID NO: 10 - protein, 234 aa, Arabidopsis thaliana
MERGGFHGYRKLSVNNTTPSPPGLAANFLMAEGSMRPPEFNQPNKTSNGGEEECTVREQDRFMPIANVIRIMRRILPAHAKISDDSKETIQECVSEYISFITGEANERCQREQRKTITAEDVLWAMSKLGFDDYIEPLTLYLHRYRELEGERGVSCSAGSVSMTNGLVVKRPNGTMTEYGAYGPVPGIHMAQYHYRHQNGFVFSGNEPNSKMSGSSSGASGARVEVFPTQQHKY

SEQ ID NO: 11 - protein, 238 aa, Arabidopsis thaliana
MERGAPFSHYQLPKSISELNLDQHSNNPTPMTSSVVVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 12 - protein, 254 aa, Oryza sativa
MEAGYPGTAANGAAADGNGGAQQAAAAPAIREQDRLMPIANVIRIMRRVLPAHAKISDDAKETIQECVSEYISFITGEANERCQREQRKTITAEDVLWAMSRLGFDDYVEPLGVYLHRYREFEGESRGVGVGVGAARGDHHHGHVGGMLKSRAQGSMVTHHDMQMHAAMYGGGAVPPPPHPPPHHHAFHQLMPPHHGQYAPPYDMYGGEHGMAAYYGGMYAPGSGGDGSGSSGSGGAGTPQTVNFEHQHPFGYK

SEQ ID NO: 13 - protein, 278 aa, Zea mays; identical to SEQ ID NO: 5

SEQ ID NO: 14 - protein, 264 aa, Zea mays
MDSSFLPAGADNGSAGGANNGGGAAQQAPPIREQDRLMPIANVIRIMRRVLPAHAKISDDAKETIQECVSEYISFITGEANERCQREQRKTITAEDVLWAMSRLGFDDYVEPLSVYLHRYREFEGEARGVGLAPAPPRGDHHHHHHSVPPSMLNKSRGPGSGAVMLPHHHHHDMHASMYGGAVPPPPHHGFLMPHPQGGHYLPYPYEPTSYGGEHALASGYYGGAAYAPGNNGGSGDGSGGSASHAPPGGSGGGFDHPHTFAYK

SEQ ID NO: 15 - protein, 262 aa, Zea mays
MNNPQNPKASAPCTLPPELPKEAVATDEAPPPMGNNNNTESATATMVREQDRLMPVANVSRIMRQVLPPYAKISDDAKEVIQECVSEFISFVTGEANERCHTERRKTVTSEDIVWAMSRLGFDDYVAPLGAFLQRMRDDSDHGGEERGGPAGRGGSRRGSSSLPLHCPQQMHHLHPAVCRRPHQSVSPAAGYAVRPVPRPMPASGYRMQGGDHRSVGGVAPCSYGGALVQAGGTQHVVGFHDDEASSSSENPPPEGRAAGSN

SEQ ID NO: 16 - protein, 194 aa, Glycine max
MRQQQVASSDQNCSNHSAAGEENECTVREQDRFMPIANVIRIMRKILPPHAKISDDAKETIQECVSEYISFITGEANERCQREQRKTITAEDVLWAMSKLGFDDYIEPLTMYLHRYRELEGDRTSMRGEPLGKRTVEYATLATAFVPPPFHHHNGYFGAAMPMGTYVRETPPNAASSHHHHGISNAHEPNARSI

SEQ ID NO: 17 - protein, 280 aa, Triticum aestivam
MENDGVPNGPAAPAPTQGTPVVREQDRLMPIANVIRIMRRALPAHAKISDDAKEAIQECVSEFISFVTGEANERCRMQHRKTVNAEDIVWALNRLGFDDYVVPLSVFLHRMRDPEAGTGGAAAGDSRAVTSAPPRAAPPVIHAVPLQAQRPMYAPPAPLQVENQMQRPVYAPPAPVQVQMQRGIYGPRAPVHGYAVGMAPVRANVGGQYQVFGGEGVMAQQYYGYGYEEGAYGAGSSNGGAAIGDEESSSNGVPAPGEGMGEPEPEPAAEESHDKPVQSG

SEQ ID NO: 18 - protein, 139 aa, Arabidopsis thaliana
MTDEDRLLPIANVGRLMKQILPSNAKISKEAKQTVQECATEFISFVTCEASEKCHRENRKTVNGDDIWWALSTLGLDNYADAVGRHLHKYREAERERTEHNKGSNDSGNEKETNTRSDVQNQSTKFIRVVEKGSSSSAR

SEQ ID NO: 19 - protein, 160 aa, Arabidopsis thaliana
MAGNYHSFQNPIPRYQNYNFGSSSSNHQHEHDGLVVVVEDQQQEESMMVKEQDRLLPIANVGRIMKNILPANAKVSKEAKETMQECVSEFISFVTGEASDKCHKEKRKTVNGDDICWAMANLGFDDYAAQLKKYLHRYRVLEGEKPNHHGKGGPKSSPDN

SEQ ID NO: 20 - protein, 179 aa, Zea mays
MADHHHHHHHGHPPDGPGGAGDQLEVIKEQDRLLPIANVGRIMKQILPPNAKISKEAKETMQECVSEFISFVTGEASDKCHKEKRKTVNGDDVCCAFGALGFDDYVDPMRRYLHKYRELEGDRAASSRGGGGGPAGAADPASASAAAGPSPSAASAGHFMFGAAMDRPDNNSSAGARPF

SEQ ID NO: 21 - protein, 171 aa, Zea mays
MAAGHHGQPPDGEDGRRAVVGGEQDRLLPIANVGRIMKQILPPNAKISKEAKETMQECVSEFIGFVTGEASDKCHKEKRKTVNGDDLCWAFGALGFDDYVDPMRGYLHKYREVEGDRAAAAASSSRGGGDHHPASASTSTSPAAAAAPGHFMFGAAAIDRPDNNTSSARPF

SEQ ID NO: 22 - protein, 166 aa, Zea mays
MSHTSNFTGFSQLEHPQPQRNSRASSSTTHDANVRHDNNLLPIANVGRIMKDALPPQAKISKHAKETIQECTTEFVGFVTGEASERCRRERRKTINGDDICHAMRSLGLDHYADAMRRYLQRYRETEELAAALNSGGGGHDGSAIQIDVRDELSIFKGSNQQGGRD

SEQ ID NO: 23 - protein, 177 aa, Oryza sativa
MADHHGGHHADGHRRQQQLQGEAADQAAAEIIKEQDRLLPIANVGRIMKQILPPNAKISKEAKETMQECVSEFISFVTGEASDKCHKEKRKTVNGDDVCWAFGALGFDDYVDPMRRYLNKYRELEGDRAAAAATSRSGAGAAAGPDHPSSSSSAAAATAGHFMFNAMDRSTDSSRQF

SEQ ID NO: 24 - protein, 185 aa, Oryza sativa
MQGLPRASSSSTSASRDRDGGDGDGGGGGVTMTNGQDNLLPIANVGRIMKDGLPPQAKISKRAKETIQECATEFISFVTGEASERCRRERRKTVNGDDVCHAMRSLGLDHYADAMHRYLQRYREGEELAASLNSSSSAAAAAAAAGSRGGGAIQIDVRAELSIFRSGNNQGKKGVNSLFVEVKLN

SEQ ID NO: 25 - protein, 102 aa, Artificial Sequence
  consensus sequence for LEC1
PGAAVVREQDRLMPIANVRIMRILPAHAKISDDAKETIQECVSEFISFVTGEANERCRERKTVNAEDVLWAMSRLGFDDYVDPLYLHRYREEGDRGAGAGVP

SEQ ID NO: 26 - protein, 402 aa, Oryza sativa
MDSLRLRPSLLAARAPGAASLPPLRRDHFLPPLCSIHRNGKRPVSLSSQRTQGPSFDQCQKFFGWKSSHHRIPHRPTSSSADASGQPLQSSAEAHDSSSIWKPISSSLDAFYALSIVSVSLLAVENLSDVSPLFLTGLLEICFVTLVQAVVAALFMNIYIVGLNQLFDIEIDKVNKPTLPLASGEYSPATGVALVSAFAAMSFGLGWAVGSQPLFLALFISFILGTAYSINLPFLRWKRSAVVAALCILAVRAVIVQLAFFLHIQATFVFRRPAVFTRPLIFATAFMTFFSVVIALFKDIPDIEGDRIFGIKSFSVRLGQKKVFWICVGLLEMAYCVAILMGATSACLWSKYATVVGHAILAAILWNRSRSIDLTSKTAITSFYMFIWKLFYAEYLLIPLVR

SEQ ID NO: 27 - protein, 411 aa, Glycine max
MDSLLLRSFPNINNASSLTTTGANFSRTKSFANIYHASSYLPNASWHNRKIQKEYNFLRFRWPSLNHHYKSIEGGCTCKKCNIKFVVKATSEKSFESEPQAFDPKSILDSVKNSLDAFYRFSRPHTVIGTALSIISVSLLAVDKISDISPLFFTGVLEAVVAALFMNIYIVGSNQLFDVEIYKINKPYLPLASGEYSFETGVTIDASFSILSFWLGWVVGSWPLFWALFEIFVLGTAYSINVPLLRWKRFAVLAAMCILAVRAVIVQLAFFLHIQTHVYKRPPVFSRSLIFATAFMSFFSVVIALFKDIPDIEGDKVFGIQSFSVRLSQKPVFWTCVTLLEIAYGVALLVGAASPCLWSKIFTGLGHAVLASILWFHAKSVDLKSKASITSFYMFIWKLFYAEYLLIPFVR

SEQ ID NO: 28 - protein, 393 aa, Arabidopsis thaliana
MESLLSSSSLVSAAGGFCWKKQNLKLHSLSEIRVLRCDSSKVVAKPKFRNNLVRPDGQGSSLLLYPKHKSRFRVNATAGQPEAFDSNSKQKSFRDSLDAFYRFSRPHTVIGTVLSILSVSFLAVEKVSDISPLLFTGILEAVVAALMMNIYIVGLNQLSDVEIDKVNKPYLPLASGEYSVNTGIAIVASFSIMSFWLGWIVGSWPLFWALFVSFMLGTAYSINLPLLRWKRFALVAAMCILAVRAIIVQIAFYLHIQTHVFGRPILFTRPLIFATAFMSFFSVVIALFKDIPDIEGDKIFGIRSFSVTLGQKRVFWTCVTLLQMAYAVAILVGATSPFIWSKVISVVGHVILATTLWARAKSVDLSSKTEITSCYMFIWKLFYAEYLLLPFLK

SEQ ID NO: 29 - protein, 199 aa, Artificial Sequence
  consensus sequence for HPT
MLSLADSLDAFYLSISVSLAVSDSPLTGLEAVVAALMNIYIVGNQLDEIKNKPLPLASGEYTGFSFLGWVGSPLFALFFLGTAYSINPLRWKRAAACILAVRAIVQAFLHIQTVRPFRLFATFMFFSVVIALFKDIPDIEGDFGISFSVLQKVFWCVLLAYVALGASWSKGHLALWSDLSKITSYMFIWKLFYAEYLLP

SEQ ID NO: 30 - protein, 404 aa, Oryza sativa
MQASSAAAAAACSAIKPAAHQHTVQVQEDKRGSEFRARFGTRKLSWGGKLSVENSALHQCQSLTRSIRRQKRQHSPVLQVRCYAIAGDQHESIATEFEEICKEVPQKLGAFYRFCRPHTIFGTIIGITSVSLLPMRSLDDFTMKALWGFLEALSSSLCMNIYVVGLNQLYDIQIDKVNKPSLPLASGEFSVATGAVLVLTSLIMSIAIGIRSKSAPLLCALFISFFLGSAYSVDAPLLRWKRNAFLAASCILFVRAVLVQLAFFAHMQQHVLKRPLAPTKSVVFATLFMCCFSSVIALFKDIPDIDGDRHFGVESLSVRLGPERVYWLCINILLTAYGAAILAGASSTNLCQMIITVFGHGLLAFALWQRAQHCDVENKAWITSFYMFIWKLFYAEYFLIPFVQ

SEQ ID NO: 31 - protein, 408 aa, Triticum aestivum
MQATTAAAAAQLLTDTRRGPRCSRARLGATRLSWPGRFAVEAFAGRCQSSATTVTHRFSAISQATSPRRKARRQCSDDQSALQAGCSKVNRDQHGYDVNWFEEISQEVSKKLRAFYQFCRPHTIFGTIIGITSVSLLPMKSIDDFTATVLKGYLEALAAALCMNIYVVGLNQLYDIQIDKINKPGLPLAAGEFSVATGVFLVVTFLIMSFSIGIHSGSVPLMYALVVSFLLGSAYSIEAPLLRWKRHALLAASCILFVRAILVQLAFFAHMQQHVLKRPLAATKSLVFATLFMCCFSAVIALFKDIPDVDGDRDFGIQSLSVRLGPQRVYQLCISILLTAYLAATVVGASSTHLLQKIITVSGHGLLALTLWQRARHLEVENQARVTSFYMFIWKLFYAEYFLIPFVQ

SEQ ID NO: 32 - protein, 270 aa, Artificial Sequence
  consensus sequence for HGGT
MQAAAAAAQRRARGLSWGVESAQRRQLQCDQEEIVKLFYFCRPHTIFGTIIGITSVSLLPMSDDFTLGLEALLCMNIYVVGLNQLYDIQIDKNKPLPLAGEFSVATGLVLIMSIGISSPLALSFLGSAYSAPLRWKRALAASCILFVRALVQLAFFAHMQQHVLKRPLATKSVFATLFMCCFSVIALFKDIPDDGDRFGSLSVRLGPRVYLCIILLTAYAAGASSTLQIITVGHGLLALWQRAHVENATSFYMFIWKLFYAEYFLIPFVQ